(19) United States
(12) Patent Application Publication
(10) Pub. No.: US 2024/0228964 A9
(43) Pub. Date: Jul. 11, 2024

(54) Highly Potent M-CENK Cells And Methods

(71) Applicant: ImmunityBio, Inc., Culver City, CA (US)

(72) Inventors: Manju Saxena, Culver City, CA (US); Syed Raza Ali, Culver City, CA (US); Greg Anderson, Culver City, CA (US); Patrick Soon-Shiong, Culver City, CA (US)

(73) Assignees: ImmunityBio, Inc., Culver City, CA (US); ImmunityBio, Inc., Culver City, CA (US)

(21) Appl. No.: 18/278,797

(22) PCT Filed: Mar. 1, 2022
(86) PCT No.: PCT/US22/18290

§ 371 Date: Aug. 24, 2023

Related U.S. Application Data
(60) Provisional application No. 63/217,097, filed on Jun. 30, 2021.
(60) Provisional application No. 63/156,269, filed on Mar. 3, 2021.

(51) Int. Cl. C12N 5/0783; A61K 35/17; A61P 35/00

(52) U.S. Cl. CPC C12N 5/0646; A61K 35/17; A61P 35/00; C12N 2501/2312; C12N 2501/2315; C12N 2501/2318; C12N 2501/39

(57) ABSTRACT

Memory-like cytokine enhanced NK (M-CENK) cells have superior cytotoxicity and can be generated/expanded from a single batch of mononuclear cells to in sufficient quantities to so form a cell-based therapeutic suitable for infusion. Advantageously, the M-CENK cells can be cryopreserved and thawed without compromising viability and cytotoxicity.

This application claims priority to our co-pending US provisional applications with the serial numbers 63/156,269, filed Mar. 3, 2021, and Ser. No. 63/217,097, filed Jun. 30, 2021, each of which is incorporated by reference herein in its entirety.

### FIELD OF THE INVENTION

The field of the invention is cell-based therapeutics and related methods therefor, especially as they relate to memory-like cytokine enhanced NK cells (M-CENK) with improved cytotoxicity and expansion characteristics.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Natural killer (NK) cells constitute a group of innate immune cells, which are often characterized as cytotoxic lymphocytes that exhibit antibody dependent cellular toxicity via target-directed release of granulysin and perforin. Most NK cells have a specific cell surface marker profile (e.g., CD3⁻, CD56⁺, CD16⁺, CD57⁺, CD8⁺) in addition to a collection of various activating and inhibitory receptors. While more recently NK cells have become a significant component of certain cancer treatments, generation of significant quantities of NK cells (and especially autologous NK cells) has been a significant obstacle as the fraction of NK cells in whole blood is relatively low.

To obtain therapeutically meaningful quantities of NK and NK-like cells, NK cells can be generated from various precursor cells. For example, various stem cell factors (SCF), FLT3 ligand, interleukin (IL)-2, IL-7 and IL-15 have been reported in various in vitro approaches to induce and expand cord blood-derived cytokine-induced killer (CIK) cells (Anticancer Research 30: 3493-3500 (2010)). Similarly, CD34⁺ hematopoietic cells can be exposed to IL-12 and other agents as is reported in US 2018/0044636. In still other approaches, human hemangioblasts were sequentially exposed to two different cytokine cocktails as described in WO2011/068896, and different cytokine cocktails were used with post-embryonic hematopoietic stem cells as taught in WO2012/128622. While at least some of these methods provide a significant n-fold expansion of NK cells, methods and reagents for such expansion are both time and resource demanding. Still further, it should be noted that many of the known methods also require NK cell culture on a feeder cell layer, which is often problematic from a technical and a regulatory perspective.

In more simplified methods, acute myeloid leukemia (AML) cells can be exposed to TpoR agonists to so induce the AML cells to form NK cells. However, such approach is likely not viable as a source for therapeutic cell preparations. Alternative methods have also relied on culturing peripheral blood cells in the presence of various interleukins, stem cell factors, and FLT3 ligands as is disclosed in WO 2011/103882. In yet another method, US 2013/0295671 teaches methods of stimulating already existing NK cells with anti-CD16 and anti-CD3 antibodies along with cytokines. While procedurally simpler, such methods still require elaborate manipulation of the cells and add significant costs due to the specific reagent required.

In still further known methods, U.S. Pat. No. 10,125,351 describes use of cord blood or peripheral blood as a source of cells that are subject to density gradient separation to isolate nucleated cells that are then cultivated with a medium that contains interferon, interleukin, a CD3 antibody and human albumin. Most advantageously, such method is amenable to perfusion culture in a bioreactor and as such significantly reduces operational difficulties. Unfortunately, the yield of NK cells is still relatively low.

Regardless of the specific manner of production, cultivated NK cells will typically not exhibit memory like character, which is particularly desirable for cancer immune therapy. In at least some attempts to produce memory like NK cells, selected NK cells were exposed to IL-12, IL-15, and IL-18 and so exposed NK cells exhibited a memory like phenotype and correlated with the expression of CD94, NKG2A, NKG2C, and CD69 and a lack of CD57 and KIR (see Blood (2012) Vol. 120, No. 24; 4751-4760). Similarly, memory like NK cells were prepared by pre-activating NK cells using various stimulatory cytokines followed by contacting the pre-activated cells with PM21 particles, EX21 exosomes, or FC21 feeder cells as is described in WO 2018/089476 and U.S. Pat. No. 10,300,089. In yet another approach to generate memory like NK cells, freshly isolated NK cells were exposed to an IL-18/IL-12-TxM fusion protein complex as is described in WO 2018/165208. While such methods typically produced at least some NK cells with memory-like character, the cytotoxicity of such activated NK cells against selected target cells was still less than optimal, possibly due to lack or low expression of specific activating receptors and/or expression of specific inhibitory receptors.

In yet another known approach cytokine induced memory like NK cells (CIML NK) can be produced in the laboratory using patient blood. However, such method is typically limited in their usage due to the relatively limited number of CIML NK cells that can be produced. Hence, multiple samples must be taken multiple times from a patient to produce enough doses to cover a complete treatment regimen. In still another approach to generate CIML NK cells as described in WO2021/006876, cord or whole blood derived mononuclear cells are activated with anti-CD16 antibodies and N-803, and then expanded using a cytokine mix. While conceptually simplified, various difficulties nevertheless remain, including presence of CD3+ cells and sub-optimal cytotoxicity against at least some target cells.

Alternatively, NK cells can be isolated from an apheresis product using beads and so isolated cells are then directly induced to produce a CIML phenotype. While such approach allows reduction in CD3+ cells in the NK cell product, that approach also limits the ability to expand the cells. Moreover, the capability of freezing and later thawing such cells back to a therapeutically effective cell product is unknown. Finally, such isolated and induced NK cells tend to have relatively lower cytotoxic potential.

Thus, even though various systems and methods of targeted antiviral therapies and vaccines are known in the art, all or almost all of them suffer from several drawbacks. Among other difficulties, many CIML NK cell preparations can prepared only with a limited number of cells and may therefore not be therapeutically effective. Moreover, and especially where the memory phenotype is induced with multiple and distinct cytokines, expense and inconsistent activities of the cytokine mixtures may prevent production of reproducible therapeutic formulations with predictable activity. Therefore, there remains a need for compositions and methods for improved NK cell-based therapies, and especially for memory-like cytokine enhanced NK cell-based compositions and methods.

### SUMMARY OF THE INVENTION

The inventive subject matter is directed to various compositions and methods of M-CENK cells and their generation and expansion, and various uses therefor. Notably, the M-CENK cells as presented herein have superior cytotoxicity, allowed for rapid and substantial expansion, and were therapeutically active after cryopreservation. Most advantageously, the M-CENK cells can be prepared from mononuclear cell-derived cytokine enhanced NK cells in a simple and effective manner in desirable quantities, and induction of the M-CENK phenotype is achieved with a single protein complex, preferably a TxM having IL-12/IL-15/IL-18 activity.

In one aspect of the inventive subject matter, the inventors contemplate a method of generating a memory-like cytokine enhanced natural killer (M-CENK) cell that includes a step of obtaining a plurality of mononuclear cells, and another step of contacting the plurality of mononuclear cells with a corticosteroid and optionally a cytokine. In still another step, the plurality of mononuclear cells are incubated in the presence of the corticosteroid and the optional cytokine to enrich the mononuclear cells in NK cells, and the enriched NK cells are then induced with a TxM fusion protein to generate the M-CENK cells, wherein the TxM fusion protein comprises a protein portion having IL-12 activity, a protein portion having IL-15 activity, and a protein portion having IL-18 activity.

In some embodiments, the plurality of mononuclear cells are cryopreserved before the step of incubating. In such embodiments, the cryopreserved mononuclear cells are preferably thawed and washed in a medium containing the corticosteroid and the optional cytokine. It is further contemplated that the step of incubating is performed over a period of between 14 and 21 days and/or until the NK cells are enriched to at least 65% of all live cells. Additionally, it is contemplated that the enriched NK cells are induced with the TxM at a concentration of between 1-25 μg/mL, typically over a period of between 12 and 16 hours.

While not limiting the inventive subject matter, it is generally preferred that the corticosteroid is hydrocortisone and that the optional cytokine is N-803. Moreover, it is also preferred that the step of incubating includes the optional cytokine. Contemplated methods will typically include a step of harvesting the M-CENK cells and formulating the harvested M-CENK cells for infusion. Where desired, the harvested M-CENK cells are cryopreserved before infusion. In further aspects of the inventive subject matter, the step of incubating is performed in an automated bioreactor.

Consequently, the inventors also contemplate a method of producing a memory-like cytokine enhanced natural killer (M-CENK) cell that includes a step of obtaining a plurality of mononuclear cell-derived cytokine enhanced NK cells (CENK), and a further step of inducing the enriched NK cells with a TxM fusion protein to generate the M-CENK cells, wherein the TxM fusion protein comprises a protein portion having IL-12 activity, a protein portion having IL-15 activity, and a protein portion having IL-18 activity.

Viewed from a different perspective, the inventors also contemplate a memory-like cytokine enhanced natural killer (M-CENK) cell produced by a method as presented herein. Most typically, the cells will be included into a pharmaceutical composition that includes a pharmaceutically acceptable carrier, which is or comprises in some embodiments a cryopreservation medium. It is further generally contemplated that the pharmaceutically acceptable carrier is formulated for infusion and/or may have a cell density of 0.5-1.5×10 7 cells/mL.

In still further embodiments, the inventors contemplate a method of treating an individual having cancer, comprising a step of administering the cells and compositions presented herein. Therefore, the compositions are also contemplated for use in the treatment of cancer. In one embodiment, the M-ceNK cells presented herein can be used, and may be administered to the patient, for killing cancer stem cells and mesenchymal cells.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 depicts CD56+CD3⁻ M-CENK cell enrichment on a bivariate dot plot. Activation of apheresis material with N-803 and hydrocortisone resulted in significant enrichment of CD56+CD3⁻ M-CENK cells.

FIG. 2 depicts comparable M-CENK enrichment kinetics from same apheresis product lot when thawed on different days.

FIG. 3 shows exemplary results for phenotyping of M-CENK cells.

FIG. 4 depicts exemplary results for cellular health markers of M-CENK cells at harvest.

FIG. 5 depicts exemplary results indicating potent cytotoxicity against tumor cells. M-CENK cell cytotoxicity was tested against two target cells including K562 and MS-1 cells using a calcein-AM based cytotoxicity assay. M-CENK cells from the different donors exhibited cytotoxicity against NK resistant cell line MS-1 in the range of 60-80% at an E:T ratio of 20:1.

FIG. 6 shows comparable results for CD56 and IFN-gamma expression on M-CENK cells produced from same lot of apheresis material but thawed at different times.

FIG. 7 depicts exemplary cytotoxicity activity of M-CENK cells against a set of target tumor cell lines.

FIG. 8 depicts exemplary activity of M-CENK cells against a set of target tumor cell lines.

FIG. 9 depicts exemplary activity of M-CENK cells against a set of target tumor cell lines.

FIG. 10 depicts exemplary IFN-γ expression of M-CENK cells.

FIG. 11 depicts exemplary cell vitality results for M-CENK cells according to the inventive subject matter.

FIG. 12 depicts further exemplary cell health results for M-CENK cells according to the inventive subject matter.

FIG. 13 depicts exemplary cytotoxicity results for M-CENK cells against MS-1 cells according to the inventive subject matter.

FIG. 14 depicts exemplary cytotoxicity results for M-CENK cells against K562 cells according to the inventive subject matter.

FIG. 15A schematically depicts an exemplary TxM, and FIG. 15B depicts the sequences for the TxM.

FIG. 16 is a graph depicting effective production of IFN-gamma by TxM induced M-CENK cells.

FIG. 17 is a graph depicting potent killing of NK-resistant MS-1 cells by TxM induced M-CENK cells.

FIG. 18 depicts results comparing cell killing of NK resistant MS-1 cells by TxM vs. cytokine cocktail induced M-CENK cells.

FIG. 19 depicts results comparing cell killing of K562 cells by TxM vs. cytokine cocktail induced M-CENK cells.

FIG. 20 depicts results for a comparison of IFN-gamma production in TxM vs. cytokine cocktail induced M-CENK cells.

FIG. 21 depicts further results for a comparison of NK specific markers on TxM vs. cytokine cocktail induced M-CENK cells.

FIG. 22 shows further results for a comparison of memory cell phenotype on TxM vs. cytokine cocktail induced M-CENK cells.

FIG. 23 illustrates lysis of Small Cell Lung Cancer by NK Cells.

FIG. 24 illustrates lysis of Ovarian Cancer by NK Cells.

FIG. 25 illustrates lysis of Breast Cancer & NSCLC by NK Cells

FIG. 26 illustrates CD56/CD16 Profile of healthy donor NK cells compared with that of ImmunityBio NK cells.

FIG. 27 illustrates ceNK and M-ceNK cells express higher levels of activating receptors NKp30, NKp44, and NKG2D.

FIG. 28 illustrates NK activating receptor expression.

FIG. 29 illustrates NK intracellular Protein Expression.

FIG. 30 illustrates NK inhibitory Receptor Expression.

FIG. 31 illustrates an overview of the M-CENK-DS manufacturing process using NANT 001 Bioreactor.

FIG. 32 shows an example of M-CENK production flow process.

FIG. 33 illustrates the potency of M-CENK lots produced by the above process.

FIG. 34 illustrates the M-CENK Surface Phenotype.

FIG. 35 illustrates that M-CENK is a potent killer of cancer cells.

FIG. 36 illustrates Apheresis Material Intermediate Stability in LN2.

FIG. 37 illustrates Cryopreserved M-CENK Cellular Product Stability.

FIG. 38 illustrates a comparison of M-CENK Production from Healthy Donor vs Patient.

FIG. 39 shows the Phase 1 protocol for clinical study QUILT-3.076, (Study of Autologous M-CENK in Subjects with Locally Advanced or Metastatic Solid Tumors)

FIG. 40 illustrates a novel fusion protein superkine (18/12/TxM) combining IL-15, IL-12 and IL-18 induces signaling via all targeted cytokine receptors. (A) Diagram showing structure of the 18/12/TxM molecule. (B-D) Freshly isolated NK cells from 3-5 healthy donors were stimulated with IL-12 (long/mL), IL-15 (50 ng/mL), or IL-18 (50 ng/mL) (IL12/15/18) or 18/12/TxM (38.8 nM) and assessed at various time intervals, gating on CD56^{bright} and CD56^{dim} NK cells. Summary data showing fold change of phosphorylated (B) STAT5, AKT, and ERK, (C) STAT4, or (D) p65 after stimulation. Data shown is mean+/−SEM and compared using paired t-test. N=3-5 human donors. (E-G) Assessment of individual cytokine activity was performed using reporter cell lines. (E) Proliferation of the IL-15-dependent 3213 cell line was assessed 3 days after incubation with various concentrations of 18/12/TxM or N-803 (F). Bioactivity was measured following incubation with various concentrations of IL-12 or 18/12/TxM with HEK12 (G). Bioactivity was measured following incubation with various concentrations of IL-18 or 18/12/TxM with HEK18 cells

FIG. 41 shows short-term activation with 18/12/TxM superkine results in NK cell activation, inducing IFN-γ and CD25 expression, and increased cytotoxicity. (A-G) Freshly isolated NK cells were activated for 16 hours with increasing concentrations of 18/12/TxM or IL-12 (long/mL)+IL-15 (50 ng/mL)+IL-18 (50 ng/mL) and assessed for the expression of indicated markers. (A) Representative flow plots showing IFN-γ and CD25 expression. (B) NK cells were incubated with varying concentrations of 18/12/TxM to identify the optimal concentration for maximal induction of CD25. (C,D) Summary data from NK cells stimulated for 16 hours with 38.8 nM 18/12/TxM. CD25 expression shown as (C) Percent CD25 positive NK cells and (D) CD25 MFI. (E) NK cells were incubated with varying concentrations of 18/12/TxM to identify to optimal concentration for maximal induction of IFN-v. (F,G) Summary data from NK cells stimulated for 16 hrs with 38.8 nM 18/12/TxM. IFN-γ expression shown as (F) Percent IFN-γ positive NK cells and (G) IFN-γ mfi. Data were compared using RM one-way ANOVA (*p<0.05, ****p<0.0001). (n=6 donors, 2 independent experiments).

FIG. 42 shows activation with 18/12/TxM superkine stimulates NK cell proliferation, similar to IL12/15/18. Purified NK cells were labeled with CFSE to track cell division and activated for 16 hours with either LD IL15 (1 ng/mL IL-15), IL12/15/16 (10 ng/mL IL12+50 ng/mL IL-15+50 ng/mL IL-18) or with 38.8 nM 18/12/TxM. After incubation, cells were washed 3 times to remove the preactivating cytokines and cultured in LD IL15. After 7 days, cells were analyzed for CFSE dilution. (A) Representative bivariate plots of both CD56 bright and CD56″ NK cells demonstrating cell division (CFSE dilution) with 18/12/TxM and IL12/15/18. (B) Summary results showing enhanced proliferation of both CD56^{bright} and CD56^{dim} NK cells 7 days after activation with 18/12/TxM or IL12/15/18, compared to low-dose IL-15 controls. Summary results are shown as the mean+/− SEM of the percentage of cells per generation (n=4 donors, 2 independent experiments). Comparisons made individually between conditions using one-way repeated measures ANOVA. Purified NK cells were ≥95% CD56⁺CD3⁻ with <0.5% CD3⁺ T cells. *P<0.05; **P<0.01; **P<0.001.

FIG. 43 illustrates multidimensional phenotype changes in NK cells is comparable after 18/12/TxM and IL12/15/18 activation. Mass cytometry analysis reveals similar changes in NK cell phenotype. Freshly isolated human NK cells were activated for 16 hrs with 18/12/TxM or IL12/15/18 and assessed for the expression of 36 markers using mass cytometry at baseline or at Day 1 or Day 6 after activation. (A) representative viSNE maps from one donor showing NK cell populations at baseline, and at one or six days after activation with IL12/15/18 or 18/12/TxM. Overlaying these populations demonstrates similar population-level changes between activating conditions. (B-C) Data reported as the average log fold change over baseline of the median expression (B) Day 1 or (C) Day 6 after activation. (n=2 donors, 1 independent experiment). R-squared value generated by simple linear regression.

FIG. 44 illustrates 18/12/TxM induces functional memory-like NK cells in vitro. (A) Functional assay schema. Briefly, NK cells from healthy donors were isolated and activated for 16 hours with LD IL15, 18/12/TxM, or IL12/15/18, washed and incubated in 1 ng/mL IL-15 for 1 week. Functional assessments were performed by stimulating the NK cells with K562 cells (E:T ratio of 5:1) or IL-12 and IL-15 and assessed for the indicated markers by flow cytometry (n=9 donors, 3 independent experiments). (B) Representative flow plot showing IFN-γ induction in K562 and IL12+IL15 stimulated NK cells. (C) Summary data showing percent IFN-γ positive NK cells stimulated with K562s or IL-12/15 as mean+/− SEM. Analysis performed using two-way ANOVA (*p<0.05, **p<0.01, ***p<0.001, ****p<0.0001). N=15 unique human donors, 6 independent experiments. (D) Percent specific killing measured by chromium release following incubation with K562 cells at various E:T ratios (n=4 donors, 2 independent experiments). Analysis performed using two-way ANOVA.

FIG. 45 illustrates 18/12/TxM activation induces a ML NK cell molecular program. (A-D) Day 1, and (E-F) Day 6 after 16-hour activation with low-dose IL-15, IL12/15/18, or 18/12/TxM. (A) Venn diagram demonstrating the number of statistically significant differentially expressed genes that were shared (purple) and distinct between low-dose and 18/12/TxM (red) and IL12/15/18 (blue) activated NK cells at Day 1 (p<0.05). (B) Scatter plot comparing the log₂ (fold change) of genes induced after IL12/15/18 or 18/12/TxM activation. (C-D) Volcano plots showing the number of differentially expressed genes between low-dose IL15 and (C) 18/12/TxM or (D) IL12/15/18 one day after activation. (E) Scatter plot showing the log₂ (fold change) of genes induced after IL12/15/18 or 18/12/TxM activation, filtered to show genes with a log₂ fold change greater than 1, or less than −1. (F) Scatter plot showing similar gene induction between conditions at Day 6. RNA sequencing analysis performed using Phantasus. Differential gene expression analysis performed using the LIMMA package. N=3 different donors per condition. Data shown is representative from 2 independent experiments using 3 donors each.

FIG. 46 illustrates 18/12/TxM induces functional memory-like NK cells in vivo, with comparable anti-tumor activity to IL12/15/18-induced NK cells. (A) Experimental design for (B) and (C). NSG mice were injected intravenously with 1×10⁶ K562-luciferase cells. After 3 days, BLI was performed to ensure leukemia engraftment. On Day 4, either control (no NK cells) or 5×10⁶ NK cells activated with low-dose IL-15, IL12/15/18, or 18/12/TxM were administered retro-orbitally to the mice. The mice were treated with rhIL-2 every other data and monitored for tumor burden (BLI). (B) representative BLI of recipient mice engrafted with K562-luc on the indicated day after tumor administration. (C) Summary of serial BLI measurements that show reduced tumor burden in mice receiving NK cells activated by IL12/15/18 and 18/12/TxM. Data presented as mean+/− SEM. Summary data are from two independent experiments with 9-10 mice per group. Differences were determined using two-way analysis of variance (2-way ANOVA). *P<0.05; **P<0.01; **P<0.001.

FIG. 47 illustrates that 18/12/TxM induces signaling via all targeted receptors at 77.6 nM. (A-C) Freshly isolated NK cells from 3-5 healthy donors were stimulated with IL-12(10 ng/mL), IL-15(50 ng/mL) and IL-18(50 ng/mL) or 18/12/TxM (77.6 nM) and assessed at various time intervals for CD56bright and CD56dim NK cells. (A) Phosphorylation of the signaling mediators downstream of IL-15 signaling, STAT5, pAKT, and pERK. (B). Phosphorylation of pSTAT4, downstream of IL-12 signaling.(C) Phosphorylation of p65, downstream of IL-18 signaling. Summary data were compared using a paired t test.

FIG. 48 illustrates that there is no difference in NK cell viability between activating conditions. Freshly isolated human NK-cells were activated with either LD IL-15 (1 ng/mL), IL12/15/18, or 18/12/TxM for 16 hrs and cultured in LD IL-15 for 7 days. Viability was assessed by flow cytometry by measuring the percent of Zombie-green negative NK cells. N=5 human donors, 2 independent experiments. Statistical analyses performed using one-way ANOVA (*P<0.05).

FIG. 49 illustrates phenotypic differences in NK cells at baseline, Day 1, and Day 6 after activation with IL-12/15/18 or 18/12/TxM. (A) Summary data demonstrating median expression of the indicated markers from FIG. 43.

FIG. 50 illustrates 18/12/TxM induces functional memory-like cells in vitro. Functional assessments were performed as described in FIG. 5 and assessed for expression of (A-C) CD107a, and (D-F) TNF. (A) Representative flow plot showing CD107a induction in K562 and IL-12+IL-15 stimulated NK cells. (B-C) Summary data showing percent CD107a positive NK cells stimulated with (B) K562s or (C) IL-12+IL-15. (D) Representative flow plot showing TNF induction in K562 and IL-12+IL-15 stimulated NK cells. (D-F) Summary data showing percent TNF positive NK cells stimulated with (E) K562s or (F) IL-12+IL-15. (n=15 donors, 7 independent experiments). Analysis performed using one-way ANOVA (*p<0.05, **p<0.01, ***p<0.001, ****p<0.0001).

FIG. 51 illustrates NL call phenotypic mass cytometry panels. The metal isotype, marker name, antibody clone, and source are shown for this mass cytometric phenotypic panel. The asterix (*) included after the source indicates antibodies were custom-conjugated using Fluidigm antibody labeling kits, per manufacturers instructions.

### DETAILED DESCRIPTION

The inventors have discovered that M-CENK cells with superior cytotoxicity can be generated that can be expanded to a desirable quantity and that can be cryopreserved and thawed without compromising functional characteristics. Notably, and in contrast to protocols that produce CIML NK cells, the present methods do not require activation of NK cells in a mononuclear cell mixture using anti-CD16 antibodies. In contrast, the methods presented herein use hydrocortisone, preferably in combination with N-803 (or IL-15) and human AB serum. Moreover, it was unexpectedly observed that enrichment and expansion can be done from cryopreserved apheresis materials, and that the M-CENK cells can also be cryopreserved and thawed without loss in cytotoxicity.

Therefore, the inventors contemplate M-CENK (memory-like cytokine enhanced NK cells) and methods of their generation, as well as cell-based therapeutics comprising such cells, and especially cryopreserved M-CENK suspensions for infusion. Viewed from another perspective, it should be appreciated that selective enrichment and expansion of NK cells from (thawed) patient apheresis material can be achieved by inclusion of hydrocortisone (and typically N-803 or other cytokine or cytokine analog with IL-15 like activity) to the growth medium to so produce high quality NK cells from the apheresis product. The so obtained cells are then activated to produce a memory phenotype. Consequently, it should be appreciated that multiple doses of high-quality M-CENK cells can be prepared from cryopreserved material without the need of repeated blood collection from donor. Indeed, M-CENK cells produced in the processes presented herein can be cryopreserved in bespoke media for an off-the shelf product, and the freeze-thaw procedure developed for cryopreservation ensures preservation of cell characteristics and viability as is shown in more detail below.

For example, in one step of contemplated methods, a cryopreserved apheresis material intermediate is produced as follows: Leukapheresis product (MNC, apheresis) from a patient are processed and cryopreserved as an apheresis material intermediate (AMI) to enable the manufacture of M-CENK product. Cryoformulation media is formulated to ensure high viability of apheresis product post-thaw. Most preferably, the cryoformulation media comprises PlasmaLyte A, 5% Albumin (Human) USP, and DMSO. Freshly prepared media is filter sterilized using 0.2 μm, a PES filter unit. Cryoformulation media is mixed with the MNC apheresis product at 1:1 ratio (by volume) to so generate the AMI. Formulated product is filled in separate Cryo-bags. 10-20 bags can be made from each apheresis product. The filled cell bags are subsequently cryopreserved to equal or lower than −85° C. using a controlled-rate freezer (e.g., CryoMed freezer).

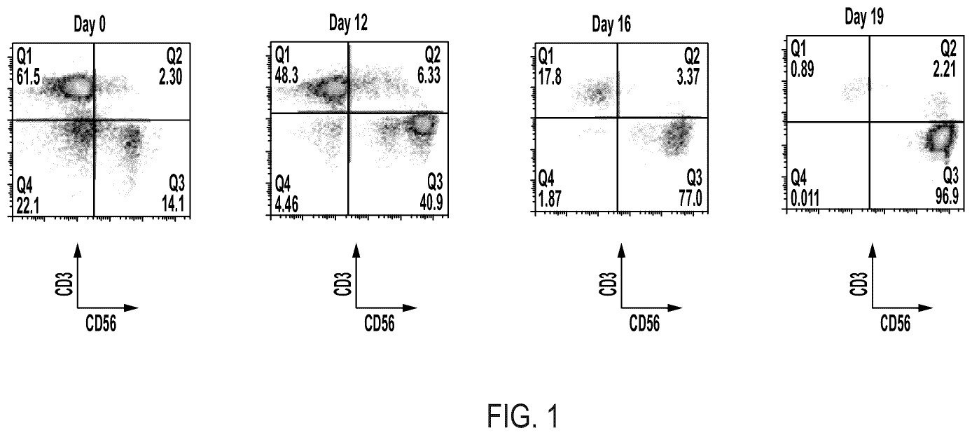

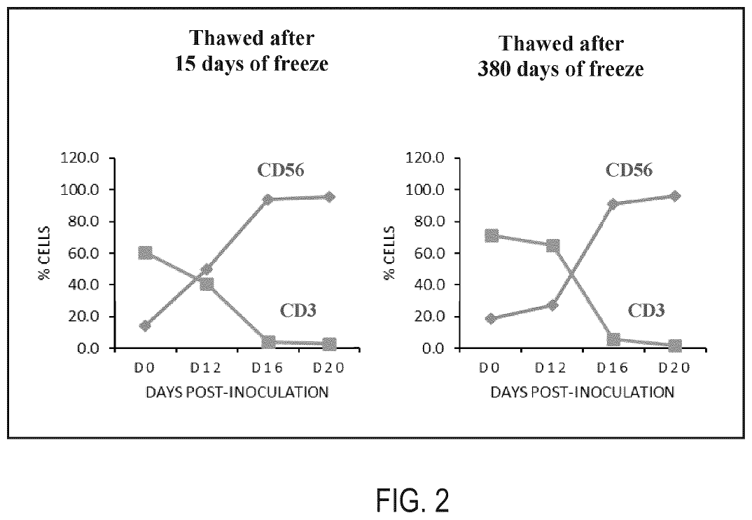

Enrichment and expansion of cytokine enhanced NK cells can then be done as follows. Cryopreserved apheresis material intermediate (AMI) is thawed in a 37° C. water bath and used for expansion. Thawed cells are washed, for example, using the Sepax C-Pro device or table top centrifuge, and re-suspended in growth media consisting of NK-MACS media containing 50-100 ng/mL N-803 and 0.2-2.0 μM Hydrocortisone and human AB serum. A strategy was developed where growth media was added to successfully enrich and expand NK cells for 20 days. For example, FIG. 1 depicts an exemplary CD56+CD3⁻ cell enrichment on a bivariate dot plot. As can be readily seen from the plot, activation of apheresis material with N-803 and hydrocortisone resulted in significant enrichment of CD56+CD3⁻ CENK cells. FIG. 2 shows that substantially identical CENK enrichment kinetics from same apheresis product lot can be achieved when thawed on different days (here: after 15 days versus after 380 days).

Generation of M-CENK cells was then performed as follows: Once the culture reaches at least 85% CD56 positive cells up to expansion day 20 at a density of at least 1×10⁶ cells/mL, fixed concentrations of N-803 (100-300 ng/mL), IL-12 (1-100 ng/mL) and IL-18 (5-250 ng/mL) are added to the culture. Cells are stimulated with the cocktail of cytokines for 14-16 hours which induces the memory like phenotype of the CD56 positive cells. After completion of the M-CENK induction stage, cells are washed using Sepax C-Pro device. Most typically, a 5% albumin (human) solution is used as washing and resuspension solution. For cryopreservation, M-CENK cells are formulated in media containing 5% Albumin (human) USP: CryoStor 10 (CS10) (1:1). M-CENK have enhanced ability to kill cancer cell targets through their increased IFN-γ production. In addition, these cells are phenotypically CD56+, CD25+, DNAM-1+, and NKP30+, NKG2D+, NKG2A+, and CD3−.

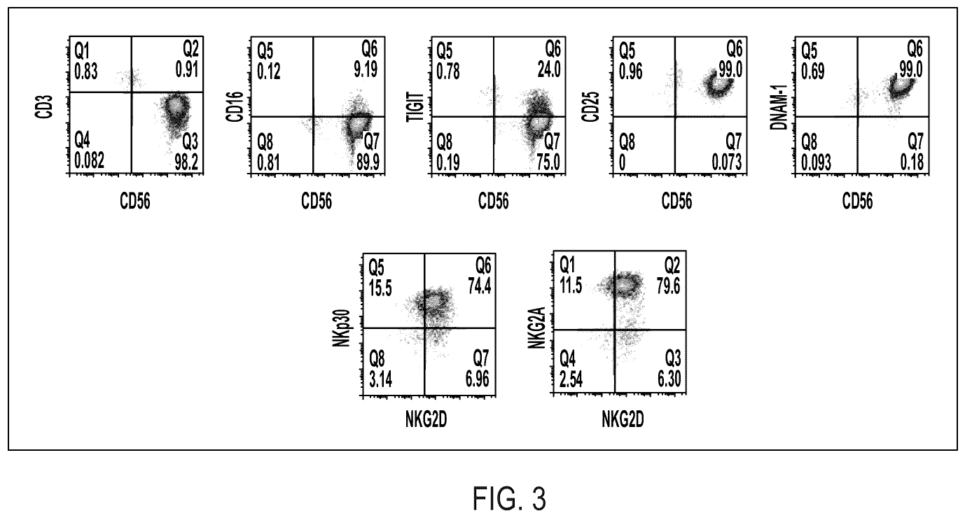

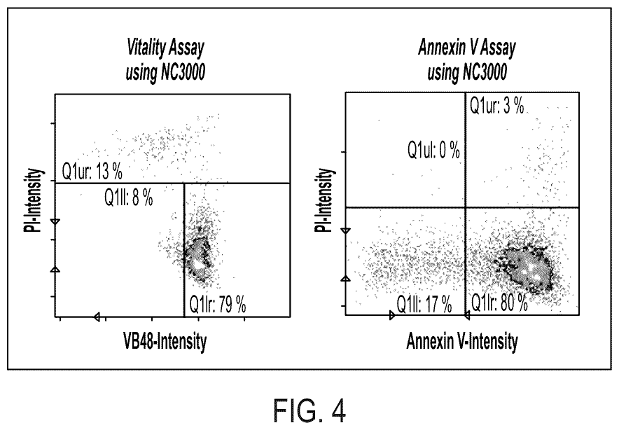

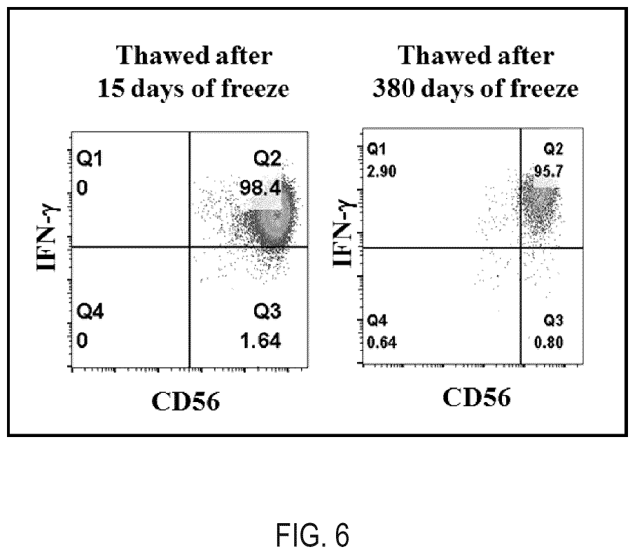

FIG. 3 provides exemplary results for phenotyping of M-CENK cells produced according to the inventive subject matter, and NK markers included DNAM-1, CD25, NKG2A, TIGIT, NKp30, CD16, and NKG2D. Moreover, the so produced cells also had high viability/vitality as can be seen from the data in FIG. 4. Likewise, freezing and thawing had no detrimental effect on IFN-γ secretion as is depicted in FIG. 6.

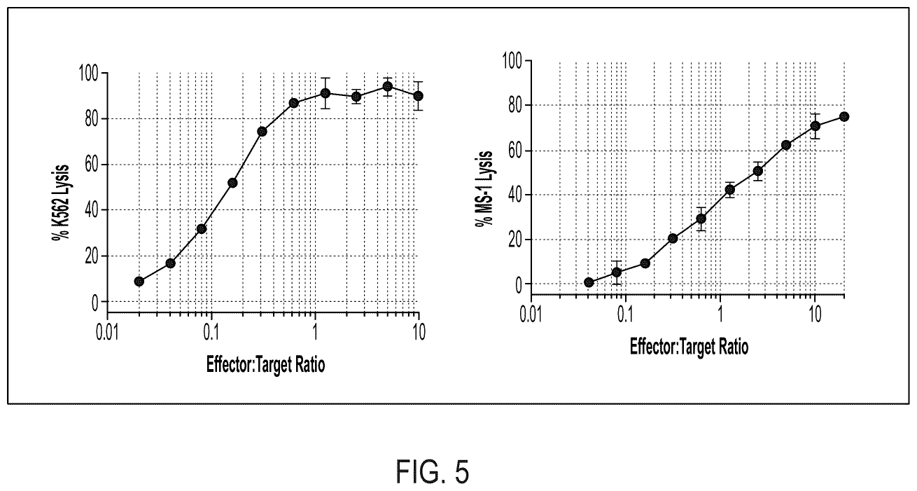

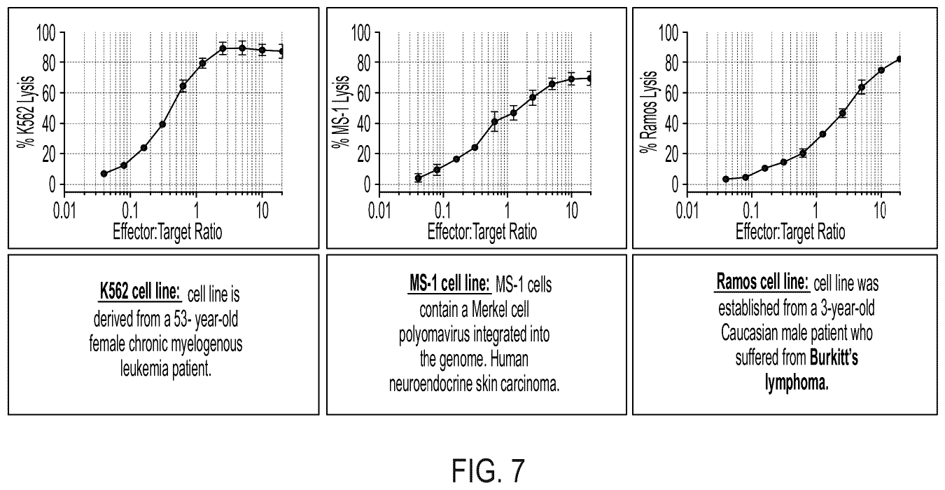

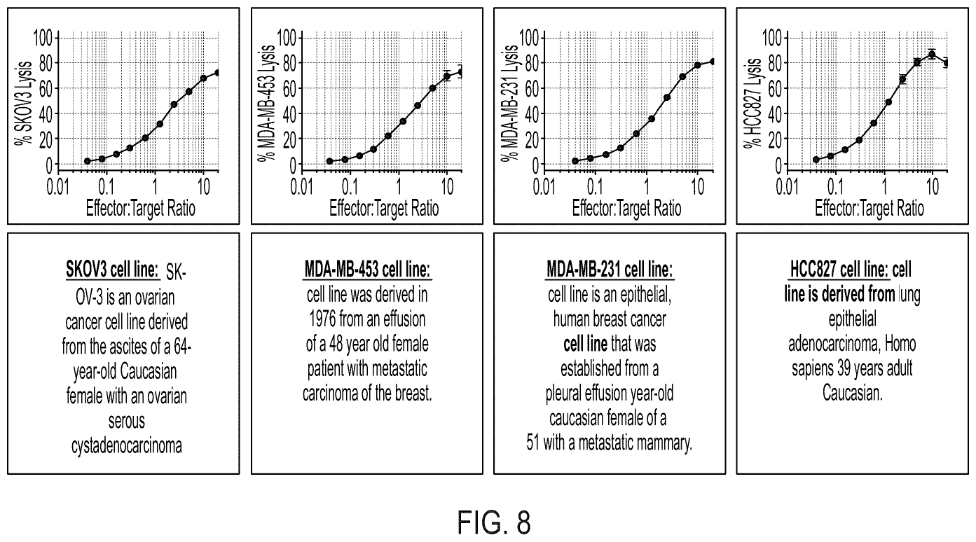

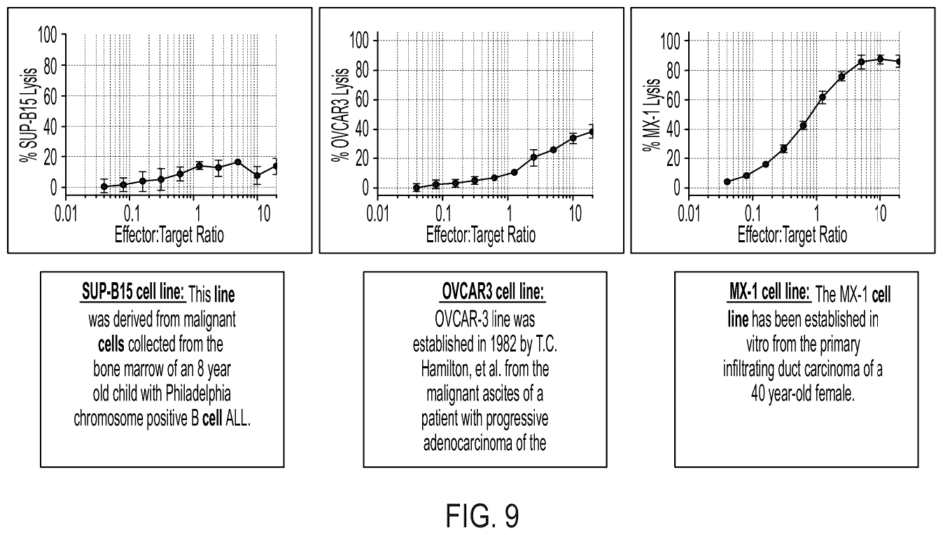

With respect to cytotoxicity, the inventors observed that the M-CENK cells had superior cytotoxic activity against a variety of target cells at a favorable effector to target ratio, even against MS-1 cells (see FIG. 5) that are known to be resistant to NK cell killing. FIGS. 7-9 depict further examples of cytotoxicity of M-CENK cells against a large variety of cancer cells. Therefore, it should be appreciated that an improved NK-cell based therapeutic product (M-CENK™, Suspension for Infusion, Cryopreserved) can be readily prepared and used, even after prolonged cryogenic storage.

Of course, it should be appreciated that numerous alternate methods or formulations can be used for freezing of apheresis material, or the apheresis material is not frozen or fresh material can be combined with previously frozen material prior to the step of enrichment and expansion. Similarly, it is contemplated that NK cells can also be purified first from whole blood, cord blood, or apheresis material and then be subjected to expansion. The so expanded cells can then be activated for memory phenotype. Likewise, it should be appreciated that while hydrocortisone is generally preferred for the step of enrichment and expansion, numerous hydrocortisone analogs and other corticosteroids (e.g., cortisol, corticosterone, cortisone, aldosterone, etc.) are also deemed suitable for use herein.

Additionally, it should be noted that the M-CENK cells can be frozen using different cryoformulation media and all known cryoformulation media are deemed appropriate for use herein. With respect to cryotreatment, it is also noted that the enriched and expanded NK cells may be frozen, and upon thawing, be subjected to activation for memory phenotype generation.

### EXAMPLES

Manufacturing Process

General Description of an Exemplary Manufacturing Process: The manufacturing process starts with the receipt of an autologous leukapheresis product (MNC, apheresis) that is processed and cryopreserved as Apheresis Material Intermediate (AMI) to enable the manufacture of product on demand. Following thaw, AMI are processed for media change using Sepax C-Pro to remove cryoformulation media first with Plasmalyte A and eluted in NK-GM containing 10% human AB serum, N-803 and hydrocortisone and seeded in NANT 001 bioreactor for expansion and enrichment to CENK. When the desired number and purity of CENK cells are generated, the cells are treated with the cytokine cocktail containing N-803, IL-12 and IL-18 cytokines to generate cytokine-induced memory-like (CIML)-NK cells as M-CENK. Post induction, cells are harvested, concentrated and washed with 5% Albumin (Human) using Sepax C Pro and eluted in 5% Albumin (Human) and then mixed with CryoStor 10 (CS10) at a 1:1 ratio to the desired VCD (0.25-0.75×10⁷ cells/mL) for a total of approximately 0.25-0.75×10⁹ M-CENK cells/bag in 100 mL volume and cryopreserved.

Apheresis to Obtain Peripheral Blood Mononuclear Cells: The manufacturing of the Autologous M-CENK cells begins following the receiving of fresh apheresis material at the manufacturing site. Following the completion of chain of custody documentation, a sample is aseptically removed from the apheresis transfer pack to enable cell viability, total nucleated cell (TNC) enumeration and phenotype characteristics determination.

Cryopreservation of the Apheresis Material Intermediate (AMI): The cryopreservation and processing steps involved in between receiving the apheresis material at the manufacturing and thawing a patient specific mononuclear cells (MNC), Apheresis material Intermediate (AMI). The cryopreservation process of AMI is initiated by determining the total nucleated cell (TNC) count and the percent viability of the fresh MNC, apheresis product.

Cryoformulation Media is freshly prepared and is filter sterilize using 0.2 μm, a PES filter unit and store on ice until used. Cryoformulation media is prepared using a mixture of PlasmaLyte A and 5% Albumin (Human) USP, and DMSO. Apheresis material is transferred to an Erlenmeyer Flask and adjusted to a desired cell density. Cryoformulation media is mixed with MNC, apheresis product at 1:1 ratio and cells are formulated to generate Apheresis Material Intermediate (AMI). Formulated product is filled in separate Cryo-bags to achieve desired cell number (2-10×10⁸ cells). Several (10-20) bags are made from each apheresis material. The filled cell bags are subsequently cryopreserved to ≤−85° C. using controlled-rate freezer (CryoMed freezer) and are then transferred to vapor phase liquid nitrogen (≤−120° C.) freezer for long-term storage.

Composition of the NK-Growth Media: The basal medium used in manufacturing of M-CENK is designated NK-Growth Medium (NK-GM). Media is prepared at the beginning of each study and then sterile filtered using 0.2 μm, a PES filter unit and stored on ice until used.

Throughout the process, different media supplements are added aseptically at specific steps of the process. (1) At the time of inoculation to NANT 001 bioreactor, thawed apheresis material intermediate (AMI) is suspended in NK-GM containing 50-100 ng/mL N-803 (0.8 nM) and 0.2-2.0 μM Hydrocortisone. (2) During subsequent media addition steps, NK-GM containing 50-100 ng/mL N-803 was added to the bioreactor. (3) Stimulation of memory phenotype is achieved by addition of NK-GM containing cytokine cocktail [N-803 (100-300 ng/mL), IL-12 (1-100 ng/mL) and IL-18 (5-250 ng/mL)]. Prior to each media addition step, the desired amount of NK-GM is mixed with fixed concentration of cytokine/supplements (N-803+hydrocortisone or N-803 alone or N-803+IL-12+IL-18), and are then filter sterilize using 0.2 μm, a PES filter unit. Prepared media is aseptically transferred to NANT 001 for cell expansion/stimulation.

Use of NANT 001 Platform for CENK Expansion: An overview of the process for the manufacture of M-CENK is illustrated below. The NANT 001 bioreactor platform system (ImmunityBio, Inc.) is a self-contained bioreactor that executes pre-programmed protocols that instruct automatic procedures and real time monitoring throughout the entirety of the recovery, enrichment, expansion, and M-CENK induction stages of the manufacturing process.

Programmable process parameters include pH monitoring, cell imaging, temperature, and rocking parameters. The NANT001 Bioreactor includes a thermostatic compartment, a touch-screen user interface, a barcode reader, a pH Estimation unit, an integrated imaging system, and a gas flow control. Components are easy to load, single-use closed-system design for safe and cGMP-compliant cell processing and include a waste bag, up to 4 L, an aseptic disconnector, a harvesting bottle, auxiliary bags ×2, up to 100 mL ea., aseptic connectors, a cell culture flask, 636 cm2, a media bag, up to 3 L, and a buffer bag, up to 3 L. Exemplary systems suitable for use herein are described, for example, in U.S. Pat. No. 10,801,005 and US 2017/0037357, incorporated by reference herein.

Thaw, Media Exchange and NANT 001 Inoculation of MNC, Apheresis—Cryopreserved Material Using Sepax C-Pro: First, the Sepax C-Pro device is initiated utilizing the CultureWash software program. Following the installation of a single use disposable kit, 1 L of Wash solution (PlasmaLyte A) and 100 mL of Resuspension solution (NK-GM containing 50-100 ng/mL N-803 and 0.2-2.0 μM Hydrocortisone) are attached to the device as per batch records. Cryopreserved apheresis material intermediate (AMI) is removed from cryostorage and the cryobag is inspected for visible signs of damage and immediately placed into a 37° C. water bath for rapid thawing.

Following thaw, a sample is aseptically removed for cell viability and TNC count determination prior to connecting the thawed cryobag material to the Sepax C-Pro device. The MNC Apheresis—Cryopreserved material is subsequently washed twice with wash solution (PlasmaLyte A) using the Sepax C-Pro device and re-suspended into a cell collection bag at ≥3.5×10⁶ cells/mL in NK-GM containing 50-100 ng/mL N-803 and 0.2-2.004 Hydrocortisone. The cell collection bag is then removed from the Sepax C-Pro device. A sample is taken to confirm cell viability and cell number, and if required the cells are adjusted to a desired inoculation cell density (1.0-5.0×10⁶ cells/mL) before transferring 50 mL of the inoculum into a NANT 001 bioreactor that contains a 100 mL of pre-warmed NK-GM containing the same concentration of N-803 (74 ng/mL) and Hydrocortisone (1 μM), in order to commence the NK cell enrichment and expansion stage. Initial cell culture volume in the NANT 001 bioreactor is 150 mL and cell density range is 0.5-1.5×10⁶ cells/mL. Multiple AMI bags are thawed if more than one NANT 001 bioreactor inoculation from same patient is planned.

Cell Recovery, Enrichment and Expansion Using the NANT 001 Bioreactor: Following inoculation of a NANT 001 bioreactor unit with 1.20-1.80×10⁸ thawed AMI cells, the culture is monitored daily using microscopy and is sampled at specific stages throughout the cell recovery, enrichment and expansion process stages. In-process monitoring (IPM) is performed to determine cell viability, cell count and the phenotype results (percentage of CD56 positive cells present). This testing is used to control the need for subsequent addition of fresh growth media containing N-803 (without hydrocortisone). Once the percentage of CD56 positive cells in the bioreactor reaches ≥85%, expansion day 20, total cell count exceeds >1×10⁶ cells/mL, the culture is transitioned into the cytokine stimulation stage of the M-CENK manufacturing process. Samples are taken for Bioburden testing before cytokine stimulation stage.

In Process Monitoring (IPM): During each media and N-803 addition, the culture is visually inspected for any sign of turbidity or visible contamination. Viable cell density, cell viability and cell phenotype analysis are performed on specified days as per the batch record to obtain the NK enrichment profile, and it is ensured that the production bioreactors are within the desired VCD specifications.

Stimulation of CENK with Cytokine Cocktail (human IL-12, IL-18 and N-803): Once the NANT 001 bioreactor culture reaches ≥85% CD56 positive cells up to expansion day 20 at a density of ≥1×10⁶ cells/mL, fixed concentrations of N-803 (100-300 ng/mL), IL-12 (1-100 ng/mL) and IL-18 (5-250 ng/mL) are added to the culture in fresh NK-GM up to a final total culture volume of approximately 650 mL. Cells are stimulated with the cocktail of cytokines for 14-16 hours which induces a memory like phenotype of the CD56 positive cells (M-CENK) contained within the NANT 001 bioreactor. This stimulation with cytokine cocktail stage in the bioreactor is then terminated by bulk cell harvesting of the culture using the NANT 001 auto-export feature. Samples are taken for mycoplasma testing prior to culture harvest.

NANT 001 Bioreactor: Cell Culture Harvest and Sepax Concentration, and Wash: After completion of the M-CENK induction stage, the NANT 001 is manually advanced to perform an automated auto-export harvest protocol. The auto-export harvest is conducted using a closed system utilizing direct sterile welds between the NANT 001 bioreactor and a collection bag. Following M-CENK export, NANT 001 bioreactor is flushed with NK-GM to retrieve any remaining cells.

After the completion of the auto-export harvest stage, the intermediate harvest bag, containing the M-CENK cells, is weighed and then directly welded to the Sepax C-Pro device for downstream processing and bulk drug substance formulation. The Sepax C-Pro from GE utilizes a single use disposable technology that allows a direct sterile weld to the BCH interim bag. Using a single use disposable kit and a designated wash and re-suspension program, the Sepax C-Pro initially performs a cell concentration step, followed by a two-chamber volume buffer exchange/wash step using 5% albumin (human) solution. Finally, the cells are concentrated and eluted into an attached 300 mL cell collection bag in an approximate volume of 50 mL of 5% human albumin solution at an anticipated cell density of 0.1-2.5×10⁷ cells/mL. The collection bag/vessel containing M-CENK is then removed from the device and QC samples are aliquoted to determine cell viability, cell density and endotoxin. M-CENK can be pooled if multiple NANT 001 bioreactors contained the same patient MNCs as the seeding inoculum.

M-CENK Drug Product Formulation: M-CENK preparation generated from multiple NANT 001 bioreactors inoculated with the same patient AMI are pooled at this stage and volume correction is made to achieve the VCD of 0.5-1.5×10⁷ cells/mL. M-CENK in 5% human albumin is subsequently mixed with equivalent volume (1:1) of the CryoStor® CS10 in a flask on ice to prepare the formulated drug product. Formulated cells are transferred into CellFreeze® infusion bags (CF-750) on ice to prepare multiple drug product bags and small QC bags. The filled infusion bags are subsequently frozen to equal or lower than −85° C. using a controlled-rate freezer. The frozen drug product is then transferred to vapor phase of LN2 freezer (equal or lower than −120° C.) for long-term storage. QC and Sterility testing is performed on QC bags at thaw. The process flow for the expansion and harvest of M-CENK cells using NANT 001 bioreactor is shown below.

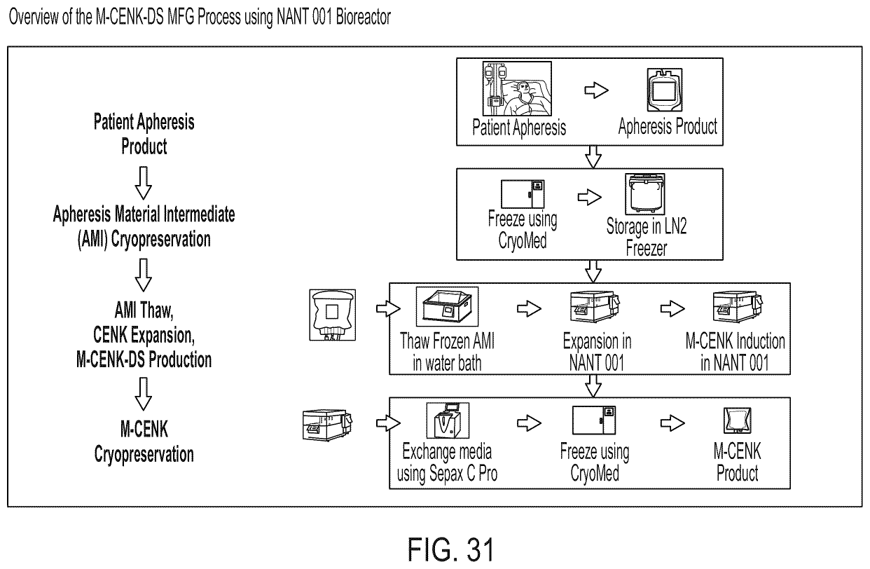

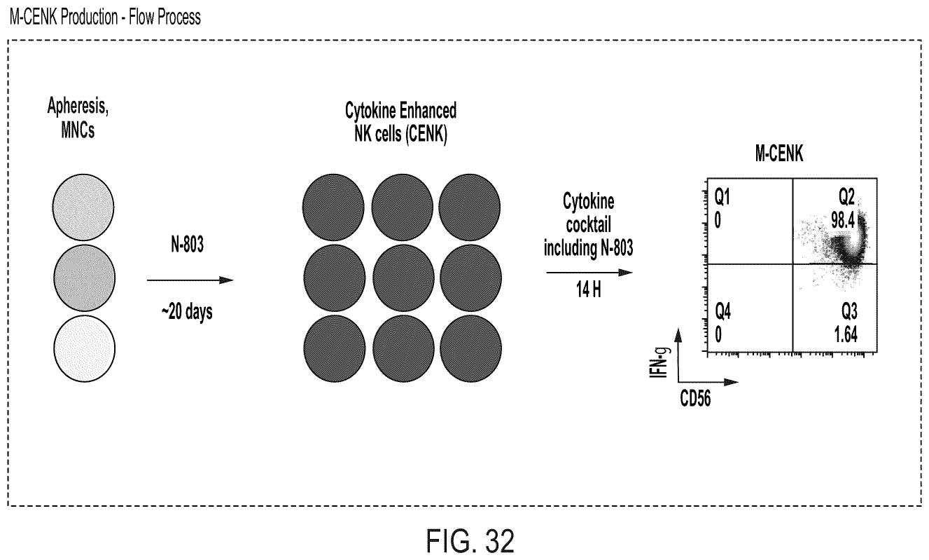

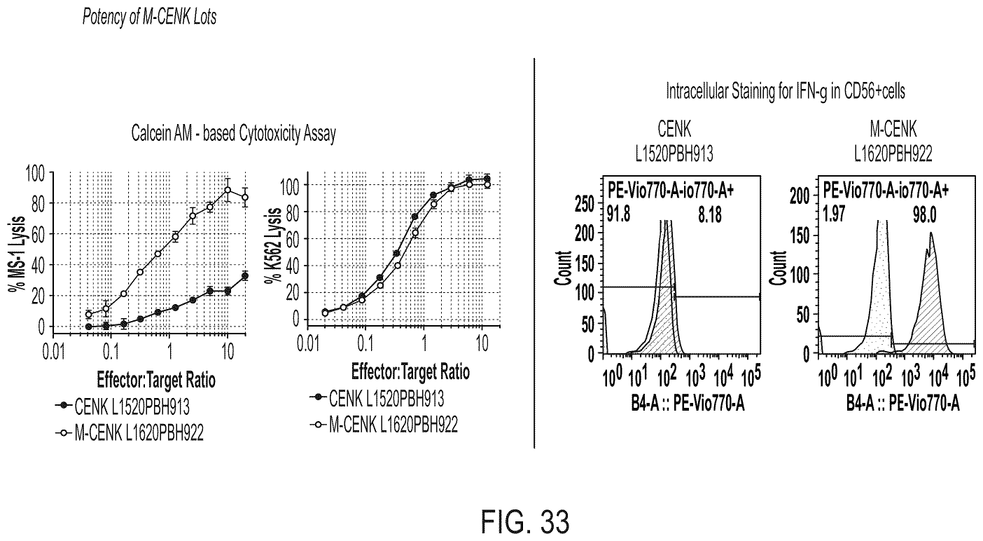

An overview of the M-CENK-DS manufacturing process using NANT 001 Bioreactor is shown in FIG. 31. FIG. 32 shows an example of M-CENK production flow process. The potency of M-CENK lots produced by this process is shown in FIG. 33.

IL-12/IL-18/N-803 Induced M-CENK

Viability and Viable Cell Density: One measure reflective of M-CENK cell structural integrity is percent viability. Viability is used as a routine in process and final product release measurement and is indicative of product quality. The following experiments describe exemplary tests to ascertain product characteristics and quality.

CD56 Expression: The neural cell adhesion molecule (NCAM1), also known as CD56, is a member of the immunoglobulin superfamily. NK cells are characterized by the expression of CD56 in the absence of CD3. PB-NK-derived M-CENK cells retain the CD56 expression.

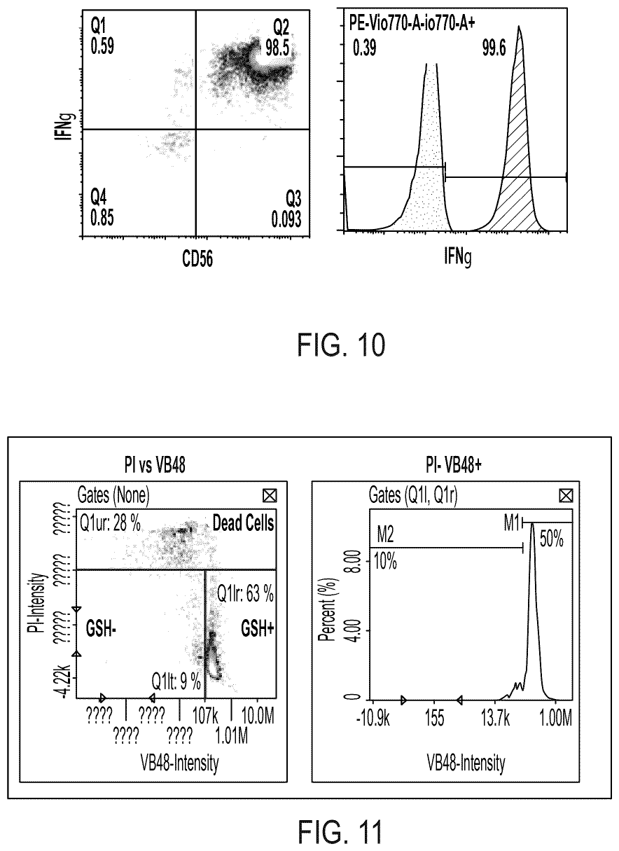

IFN-γ Expression: NK cells are cytolytic and cytokine-producing effector cells of the innate immune system. They are a major source of IFN-gamma (IFN-γ) that interferes in tumor activity. A flow cytometry based intracellular cytokine staining assay was used to analyze IFN-γ production. As can be seen from FIG. 10, Significant amount of IFN-γ was detected in M-CENK cells that were generated by NANT 001 process and expression was highly homogeneous (99.6% of CD56+ cells were positive for IFN-γ staining and displayed an MFI of 15759 (red) vs 122 non-stained sample (blue).

Phenotype Analysis: (A) DNAM-1: is a cell surface glycoprotein that functions as an adhesion molecule to synergize with activating receptors and trigger NK cell mediated cytotoxicity. DNAM-1+ve NK cells produces higher IFNγ than their DNAM-1-ve counterparts following stimulation with IL-12 and IL-18. DNAM-1 is upregulated on M-CENK cells as observed in the phenotyping assay described below. (B) TIGIT: is a checkpoint receptor that may negatively influence NK cell cytotoxicity activity. M-CENK generation showed no significant change in TIGIT expression. TIGIT expression was analyzed in the phenotyping assay described below. (C) CD25: Natural killer cells express IL-2Rα—chain (p55), identified as CD25 for the formation of the high-affinity IL-2R. CD25 is upregulated on M-CENK cells. (D) CD16: is present on select CD56+ peripheral blood NK cells. Upon recognition of antibody-coated cells it delivers a potent signal to NK cells, which eliminate targets through direct killing and cytokine production.

GSH Cell Vitality: One measure reflective of M-CENK cell health status is the intracellular reducing power available to the cell or percent vitality. Expression of intracellular reduced thiols (glutathione; GSH) can be analyzed by staining the cell line with a specific dye (VitaBright-48™, VB48) which reacts with thiols forming a fluorescent product in combination with Acridine Orange (AO) and Propidium Iodide (PI) to stain nucleated cells and dead cells, respectively. The stained sample is subsequently analyzed by NucleoCounter® NC3000™ imaging cytometer. M-CENK cells from different culture batches expanded in NANT 001 bioreactor displayed characteristics of healthy cells with high vitality (GSH+ve, PI-ve) and were comparable to each other as can be seen from the exemplary results in FIG. 11.

Annexin V Cell Health: Another measure reflective of M-CENK cell health is the presence of apoptotic, pre-apoptotic and necrotic cells in culture. Annexin V assay enables detection of translocalisation of phosphatidylserine to the outer cell membrane layer indicating early apoptosis. Quantification of early apoptotic cells can be achieved by staining the cells with an Annexin V-AF488 conjugate along with Hoechst 33342 and PI to stain nucleated cells and dead cells, respectively. The stained sample is subsequently analyzed by NucleoCounter® NC3000™ imaging cytometer. M-CENK cells from different culture batches expanded in NANT 001 bioreactor displayed characteristics of a healthy cells (Annexin negative PI negative) and were comparable to each other, and exemplary results are shown in FIG. 12.

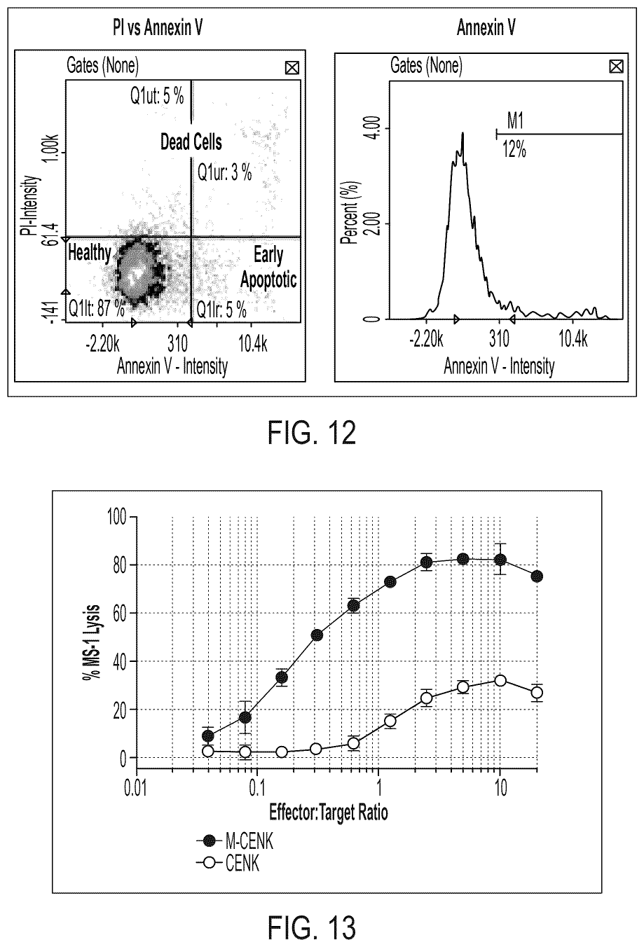

M-CENK Cytotoxicity Against MS-1 Cells: An important functional assay used to measure the activity of M-CENK cells is to assess cytotoxicity against the MS-1 target cell line, a cell line that is relatively resistant to general NK cell cytotoxicity. As an example of extended characterization, the graph in FIG. 13 below shows the results of the M-CENK cell (red) cytotoxicity depicted graphically over a wide range of Effector to Target (E:T) cell ratios. Control CENK cells (blue) were also expanded in the NANT 001 bioreactor but not induced for M-CENK generation.

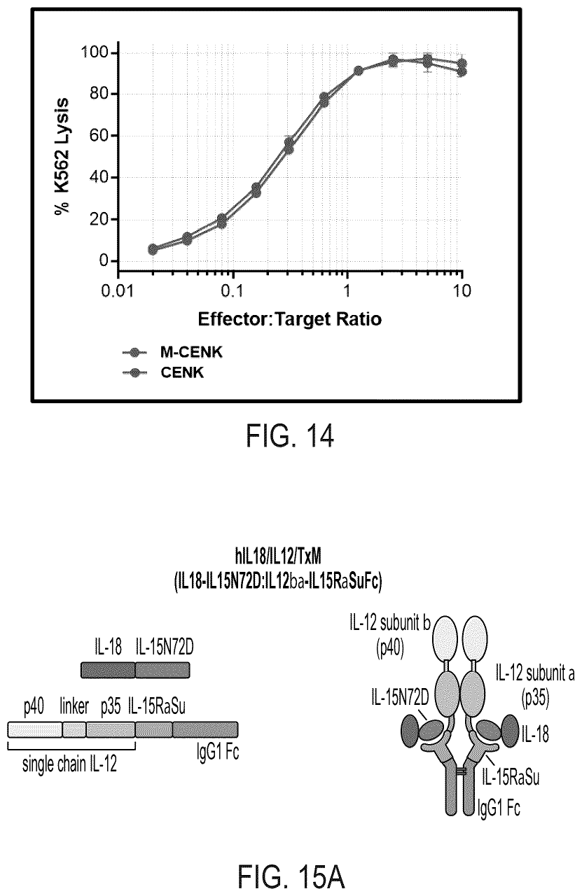

Cytotoxicity of M-CENK Cells Against K562 Cells: Testing for the natural cytotoxicity of M-CENK cells towards the K562 cell line is part of extended characterization of M-CENK cells. The graph in FIG. 14 presents the results comparing the natural cytotoxicity of M-CENK cells (red) against K562 cells vs the Control CENK cells (blue) that were also expanded in the NANT 001 bioreactor but not stimulated with cytokine cocktail.

TxM Induced M-CENK Cells

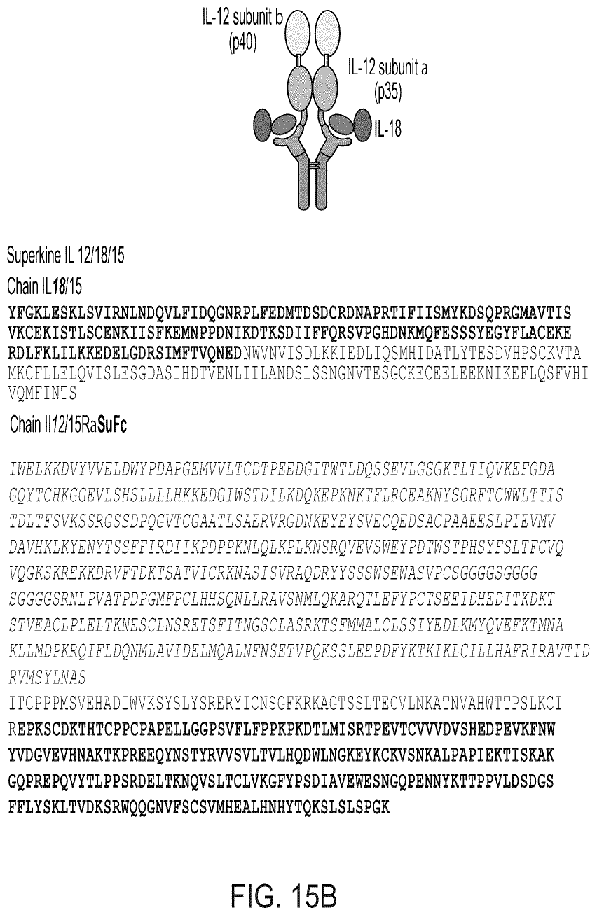

The TxM used in the studies below was obtained from ImmunityBio, Inc. and is a fusion protein as shown in FIG. 15A comprising N-803, IL-12 and IL-18, and FIG. 15B depicts the sequences used in the TxM. This superkine was evaluated as a replacement for the cytokines N-803 in which IL-18 was fused to the IL-15 portion of the N-803, and in which a IL-12 single-chain heterodimer of IL-18 was fused to IL-15 Receptor alpha portion of N-803, in their ability to induce NK memory phenotype.

The production of CENK from mononuclear cells was described as above, however, in the present process the induction/stimulation of CENK with the cytokine cocktail (human IL-12, IL-18 and N-803) was replaced by induction/stimulation with a TxM as described immediately above. In this context, it should be noted that the TxM used herein had equimolar ratios of IL-15 analog (N-803) to IL-18 to IL12 (single chain) of 1:1:1. Notably, the molar ratio of components in the TxM is substantially distinct from the molar ratio of the cytokine cocktail.

Moreover, it should be noted that the TxM had all three cytokine function associated in close proximity and as such provides contemporaneous activation, whereas induction/stimulation of CENK with a cytokine cocktail (human IL-12, IL-18 and N-803) will allow for spatially and temporally separate activation events. Surprisingly, the TxM allowed for substantially identical, If not even improved formation of M-CENK as compared to use of the cytokine cocktail. In addition, as the TxM is provided as a single protein complex and requires only a single addition to the CENK cells (as opposed to three additions), the risk of contamination is significantly reduced. Furthermore, inconsistencies in potency of individual cytokine preparations (e.g., lot-to-lot variations) can be entirely avoided as the TxM is provided as a single protein complex.

For comparison with the TxM induced cells, N-803 enriched NK cells (CENK, Cytokine-enhanced NK cells) were incubated with cytokine cocktail containing fixed concentrations of N-803 (175 ng/mL), IL-12 (10 ng/mL) and IL-18 (50 ng/mL) or with TxM (9.8 μg/mL) alone. Cells were stimulated for 14-16 hours, which induced a memory like phenotype of the CD56 positive cells (M-CENK) as is shown in more detail below.

Post-harvest, M-CENK cells from both treatment experiments were evaluated in various assay for memory cell characteristics. Cytokine priming is generally required for NK cell proliferation and function. However, cytokines may also lead to a dose dependent death of NK cells. Viability of N-803 expanded NK cells was therefore evaluated before and after TxM stimulation. As can be seen form the Table below, TxM induced M-CENK cells showed high viability that was comparable to the CENK cells (>90%).

| Cell Type | % Viability |
|---|---|
| CENK | >90% |
| Cytokine cocktail induced M-CENK | >90% |
| TxM for 14 hour | >90% |

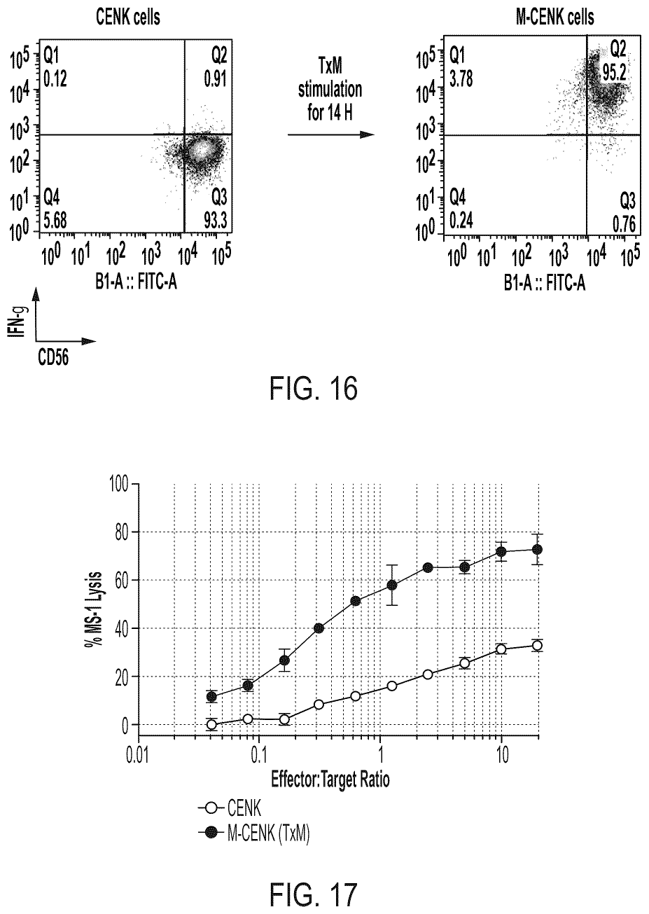

M-CENK cells are major IFN-gamma producers. To evaluate if TxM induced M-CENK cells develop IFN-gamma producing capacity, a flow cytometry based staining method was employed. As can be seen from FIG. 16, significantly higher numbers of IFN-gamma expressing cells were observed in M-CENK cells as compared to CENK cells. Here, the graph depicts exemplary results demonstrating that TxM induced M-CENK cells are effective producers of IFN-gamma as pro-inflammatory cytokine.

With respect to cytotoxicity, various experiments were performed to establish that TxM induced M-CENKS cells had significant cytotoxicity against various cell lines.

In one set of experiments, TxM induced M-CENK cell cytotoxicity was tested against MS-1 cell. MS-1 is a skin carcinoma cells with a resistance to general NK cell cytotoxicity. Cytotoxicity of M-CENK cells against MS-1 cell line was measured over a wide range of Effector to Target (E:T) cell ratios in the cytotoxicity assay. Notably, M-CENK but not CENK cells induced significant lysis of MS-1 cells, suggesting that the generated product gained potent cytotoxicity activity against resistant tumor cells. In particular, FIG. 17 demonstrates that TxM induced M-CENK cells are potent killer of NK-resistant MS-1 cells as can be readily taken from the graph.

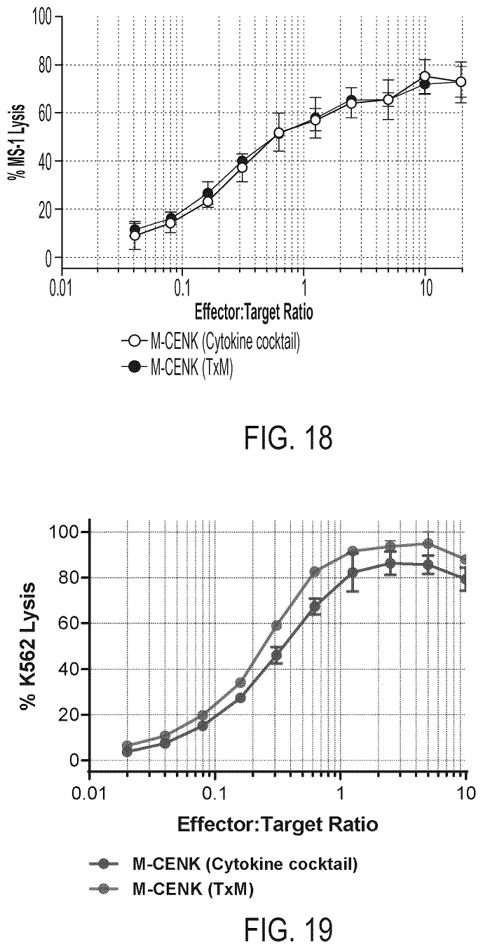

M-CENK cells from both treatments were then compared in a cytotoxicity assay. Here, cells produced from either treatment induced potent and comparable cytotoxicity against MS-1 suggesting that the TxM can serve a replacement for the cytokine cocktail. FIG. 18 depicts exemplary results for a comparison of TxM vs. cytokine cocktail induced M-CENK cells to kill NK resistant MS-1 cells.

To evaluate if TxM induced M-CENK cells retain NK cell natural cytotoxic activity, M-CENK cells were mixed with K562 target cells in a calcein-based cytotoxicity assay. Effector and target cells were mixed together at varying ratio. M-CENK produced from either treatment induced potent and comparable cytotoxicity against K562, suggesting that the NK cell natural cytotoxicity was retained in the activation process using TxM. FIG. 19 shows typical results for a comparison of the ability of TxM versus cytokine cocktail induced M-CENK cells to kill K562 cells.

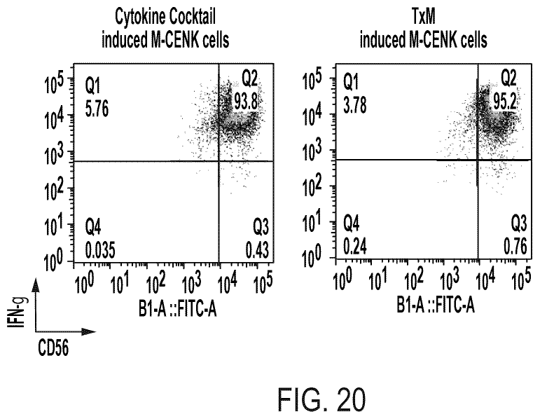

In a further experiment, M-CENK cell from both treatments (TxM induced and cytokine cocktail induced) were compared for their potential to produce IFN-gamma. Remarkably, M-CENK generated from either treatment induced potent IFN-gamma as observed in the flow cytometry-based assay. In particular, FIG. 20 depicts exemplary results for a comparison of IFN-gamma production in TxM and cytokine cocktail induced M-CENK cells.

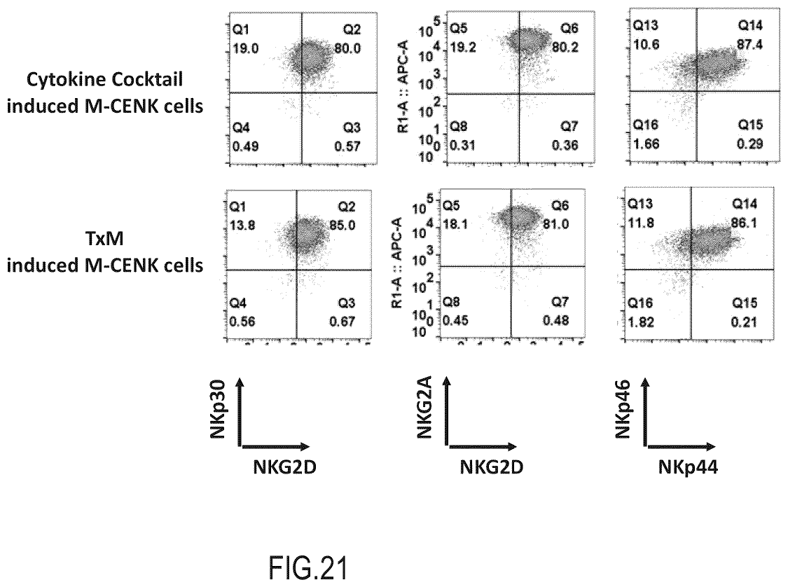

It is well known that NK cell activating receptors play a key role in triggering the anti-tumor response of NK cells. The inventors therefore investigated whether TxM treatment would influence the expression of one or more NK-specific receptors. Notably, comparable expression of NKG2D, NKp30, NKp44, NKG2A, and NKG2C were observed on cells generated from either treatment as is shown in the exemplary results of FIG. 21. Here, the graphs show typical results for a comparison of NK specific markers on TxM and cytokine cocktail (IL-12+IL-18+N-803) induced M-CENK cells.

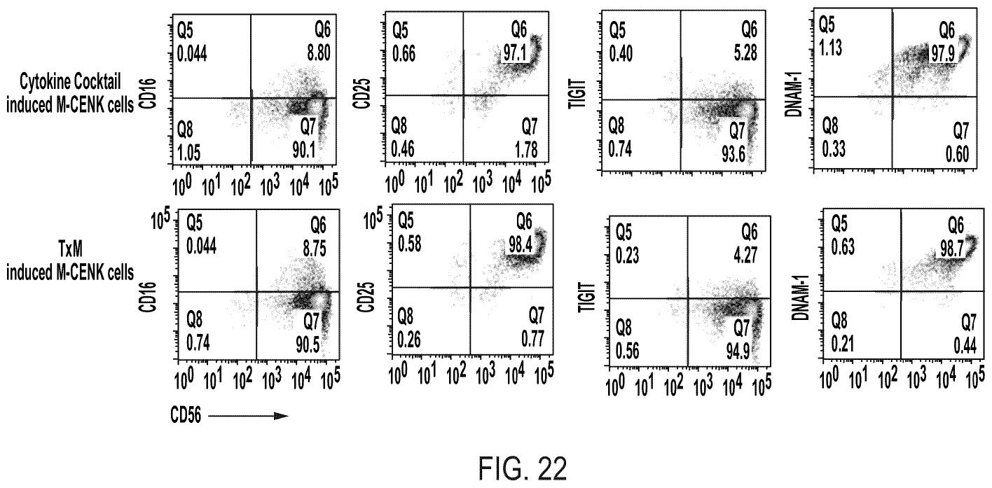

To establish that the TxM-treated CENK cells will indeed differentiate to an M-CENK phenotype, the inventors assayed the treated cells for the presence of specific memory-type associated markers. More specifically, differentiation of CENK to M-CENK is generally accompanied by changes in specific receptors expression including DNAM-1 (DNAX Accessory Molecule-1), CD25, and CD16. To that end, changes in the expression status of these receptors was detected using a flow cytometry-based assay. Once more, M-CENK cells from either treatment showed comparable expression of these receptors. FIG. 22 shows exemplary results for a comparison of memory cell phenotypes for TxM and cytokine cocktail induced M-CENK cells.

In view of the above, the experimental results clearly and unexpectedly demonstrate that a TxM fusion protein can serve as a replacement for an IL-12/IL-18/N-803 cocktail for induction of an NK memory phenotype in CENK cells (M-CENK). These cytokines may also serve as a replacement for other NK cell activating cytokines (e.g., IL-21) that often increase cytotoxicity but will cause apoptosis at high concentrations.

M-ceNK Cells Provide Enhanced Cytotoxic Effect

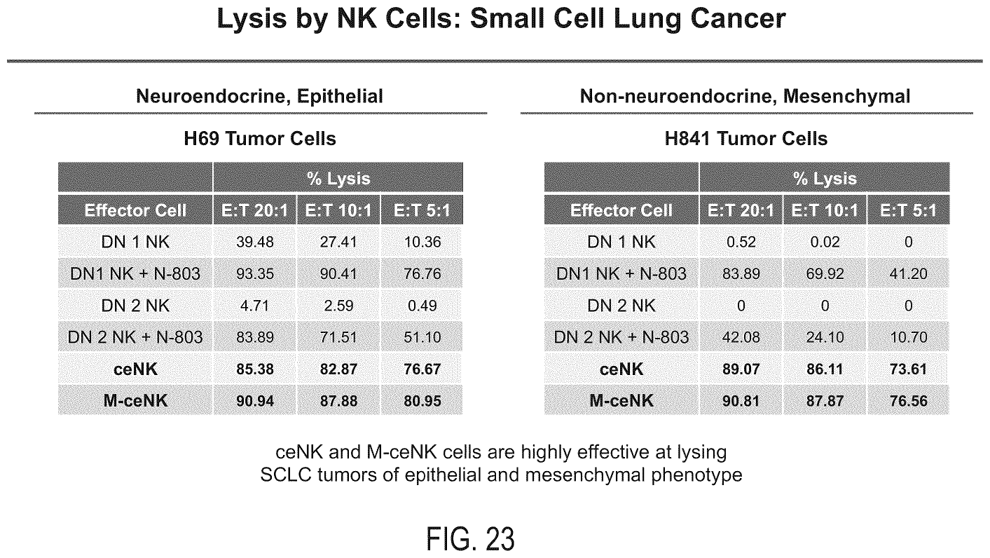

The inventors also discovered that where various NK cells were treated with an N-803 (or a TxM having an IL15 receptor scaffold as presented herein) containing cytokine cocktail, such cells had superior cytotoxicity against a variety of cancer cells, and even against those that had undergone EMT (endothelial to mesenchymal transition). Notably, thusly treated cells exhibited a phenotype that was CD56^{bright}, CD16^{low}, had high NKp44 expression, and low TIGIT expression relative to untreated cells. FIGS. 23-30 provides exemplary results for such improved cytotoxicity. Moreover, and in view of the above findings it is also contemplated that T cell diversity can be achieved in a similar manner.

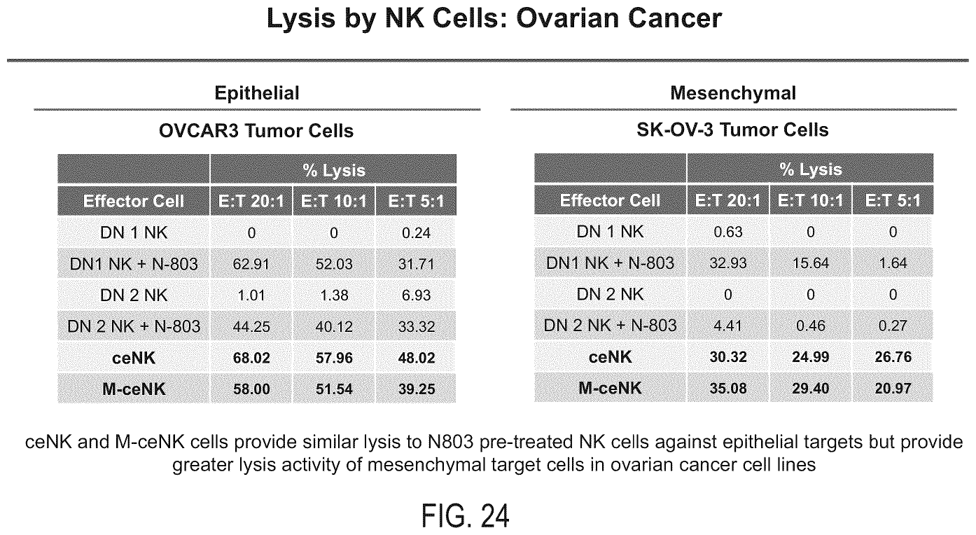

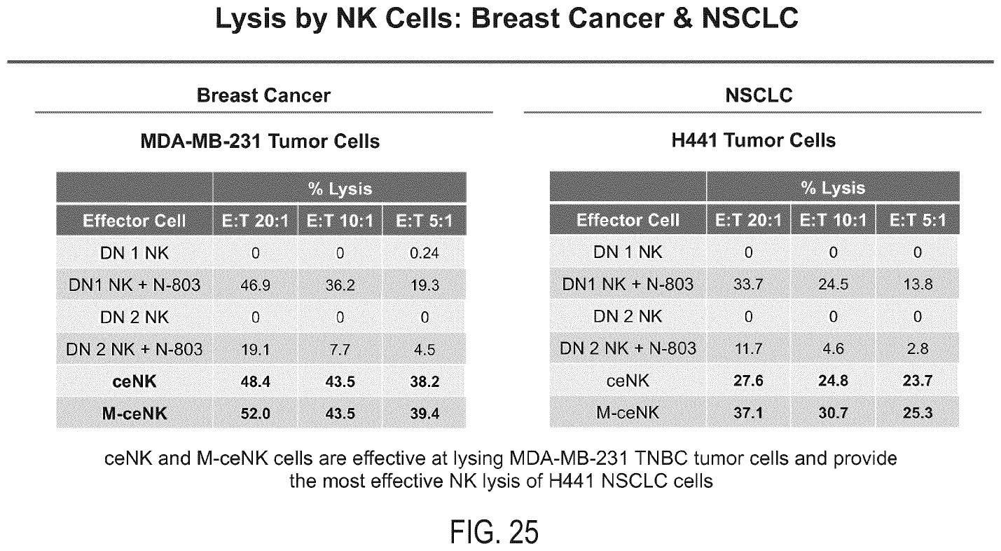

The NK cell lysis assay comprised NK Cell Effectors such as ceNK, M-ceNK, Healthy donor NK cells (2 donors), and Healthy donor NK cells pre-treated with N-803 (2 donors). The tumor cells comprised SCLC—H69 & H841, Ovarian—OVCAR3 & SK-OV-3, Breast—MDA-MB-231, NSCLC—H441. NK and tumor cells were co-cultured for 6-hour duration, cell counts collected via Celigo system. E:T ratios of 20:1, 10:1, and 5:1 assessed. The results are shown in FIGS. 23-25 respectively. FIG. 23 illustrates lysis of Small Cell Lung Cancer by NK Cells. The results show that ceNK and M-ceNK cells are highly effective at lysing SCLC tumors of epithelial and mesenchymal phenotype. FIG. 24 illustrates lysis of Ovarian Cancer by NK Cells. The results show that ceNK and M-ceNK cells provide similar lysis to N803 pre-treated NK cells against epithelial targets but provide greater lysis activity of mesenchymal target cells in ovarian cancer cell lines. FIG. 25 illustrates lysis of Breast Cancer & NSCLC by NK Cells. ceNK and M-ceNK cells are effective at lysing MDA-MB-231 TNBC tumor cells and provide the most effective NK lysis of H441 NSCLC cells. These results show that the M-ceNK cells disclosed herein may be used as a treatment of cancer stem cells and Mesenchymal stem cells.

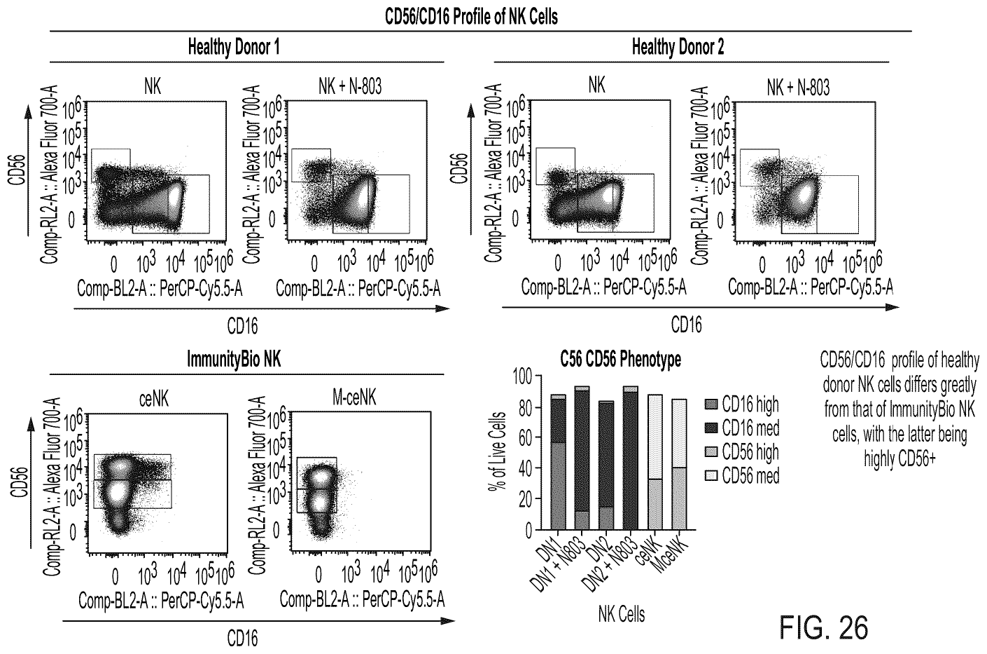

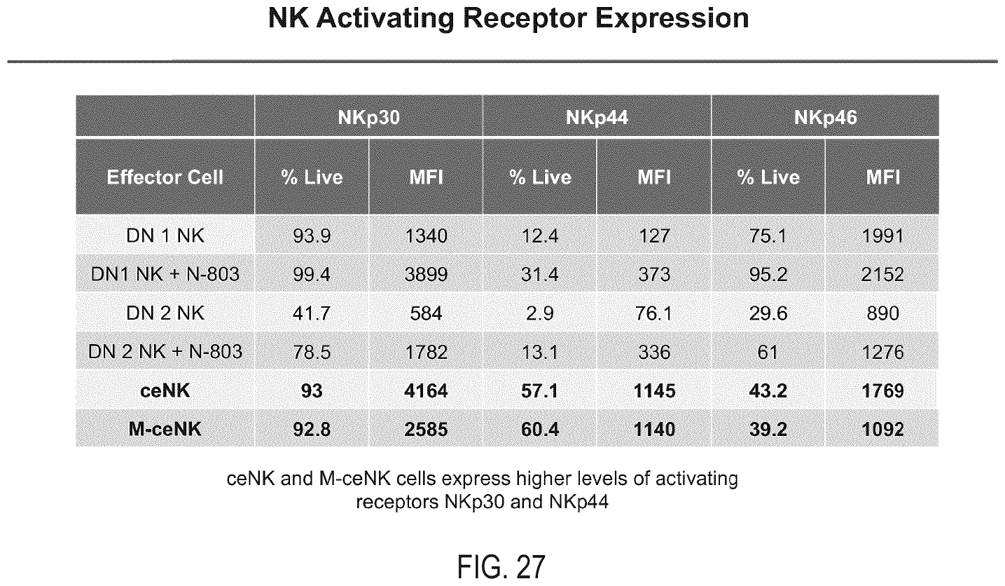

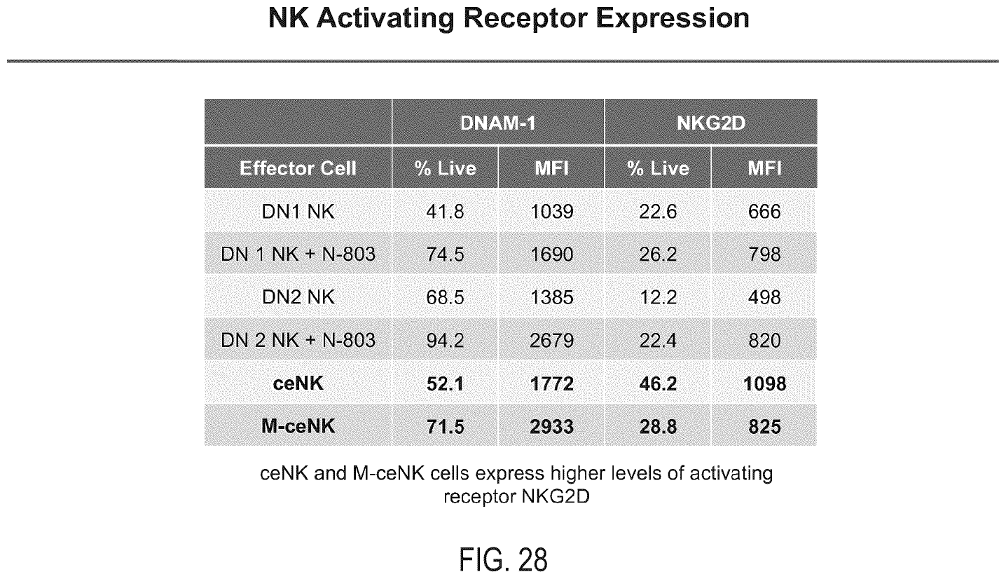

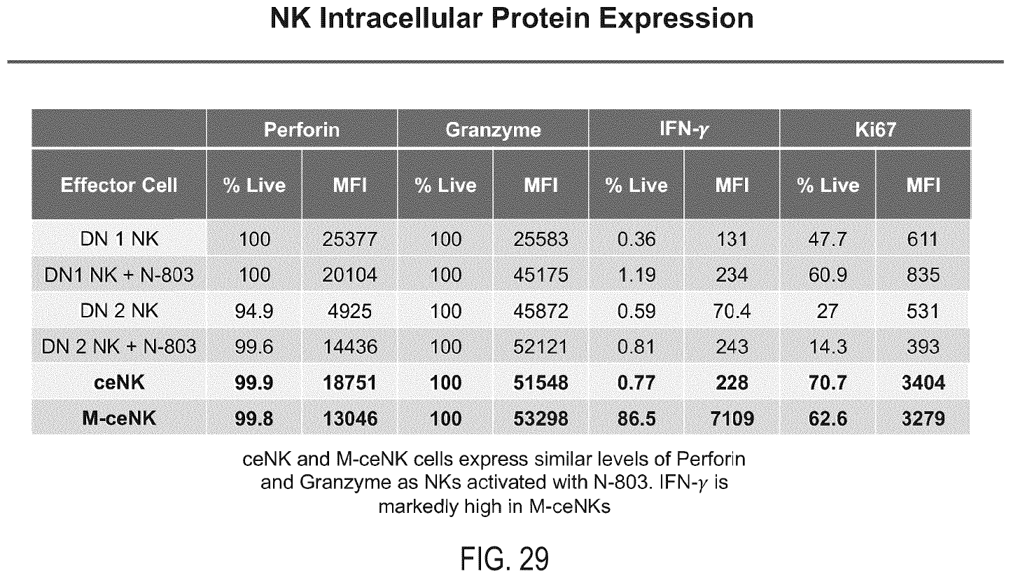

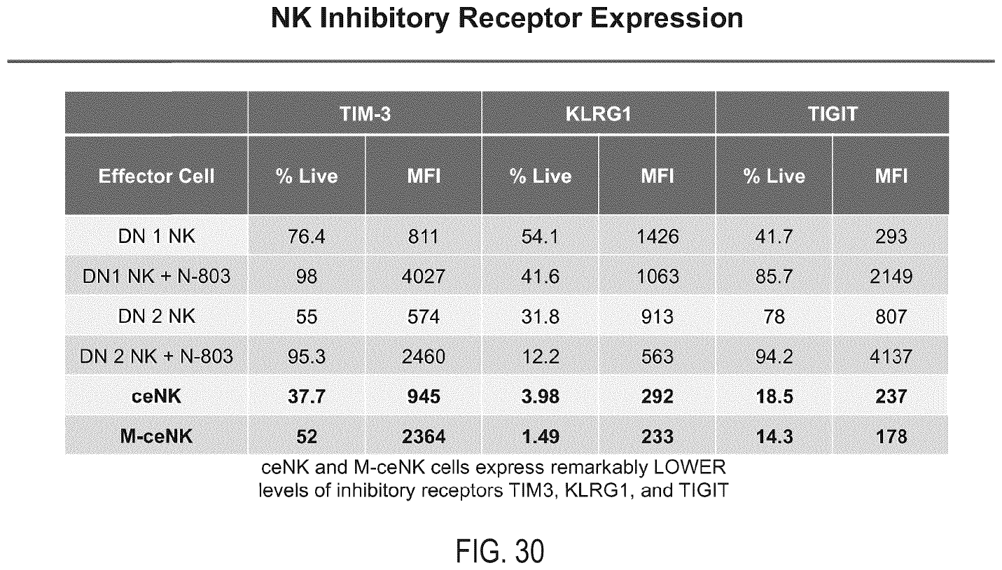

An overview of NK receptors assessed via flow cytometry may be found in Chan. C et al, Cell Death & Differen., 2016, which is incorporated by reference herein in its entirety. CD56/CD16 Profile of NK Cells-CD56/CD16 profile of healthy donor NK cells differs greatly from that of ImmunityBio NK cells, with the latter being highly CD56+, as shown in FIG. 26. ceNK and M-ceNK cells express higher levels of activating receptors NKp30, NKp44, and NKG2D as shown in FIG. 27-28. FIG. 29 illustrates NK Intracellular Protein Expression. ceNK and M-ceNK cells express similar levels of Perforin and Granzyme as NKs activated with N-803. IFN-γ is markedly high in M-ceNKs. FIG. 30 illustrates NK Inhibitory Receptor Expression. ceNK and M-ceNK cells express remarkably lower levels of inhibitory receptors TIM3, KLRG1, and TIGIT.

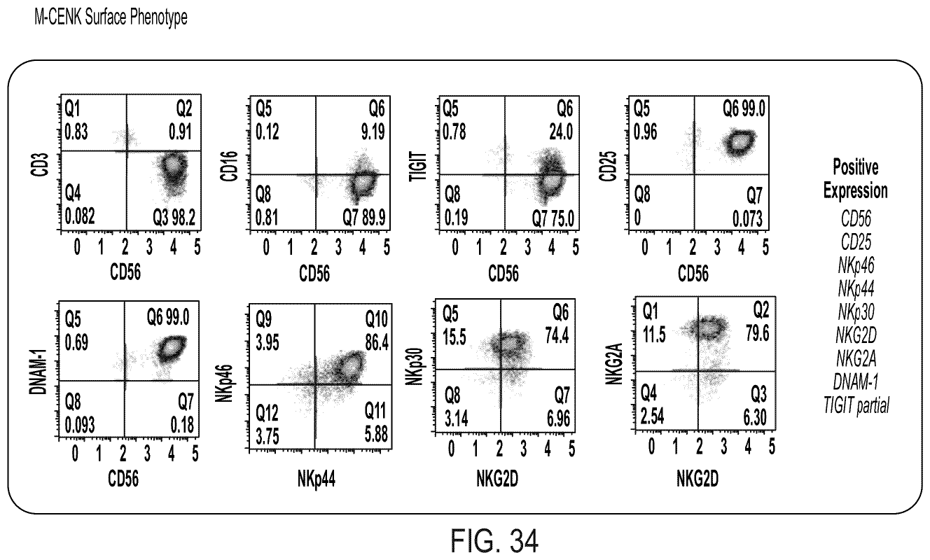

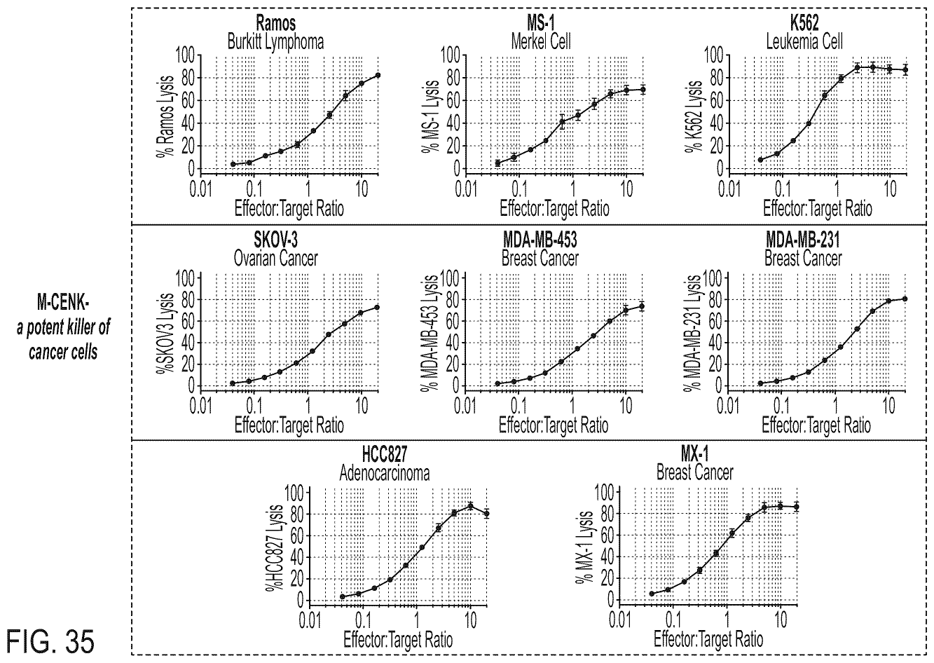

FIG. 34 illustrates the M-CENK Surface Phenotype. As shown therein, a positive expression was seen for CD56, CD25, NKp46, NKp44, NKp30, NKG2D, NKG2A, DNAM-1, and TIGIT. The effect of M-CENK on various types of cancer cells is shown in FIG. 35, which illustrates that M-CENK is a potent killer of cancer cells.

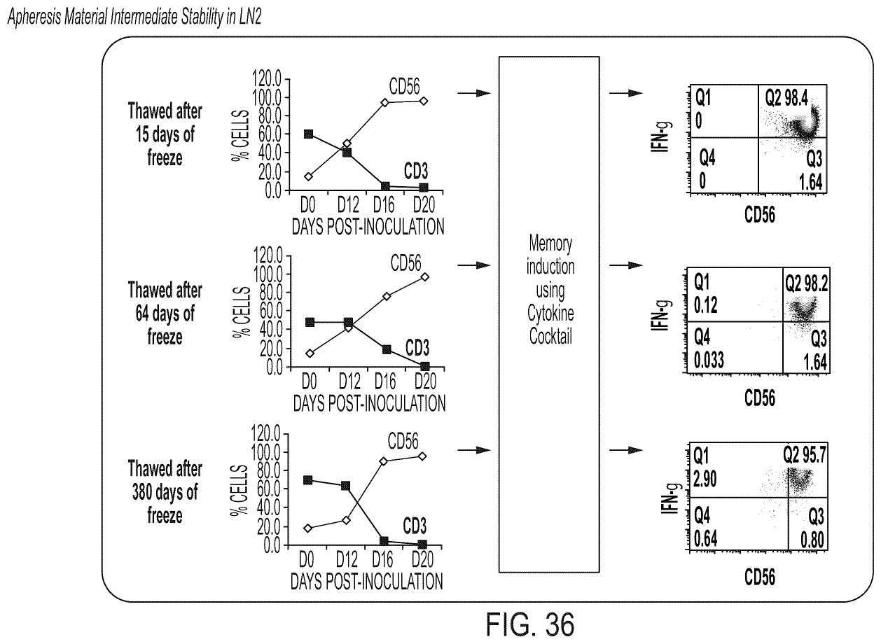

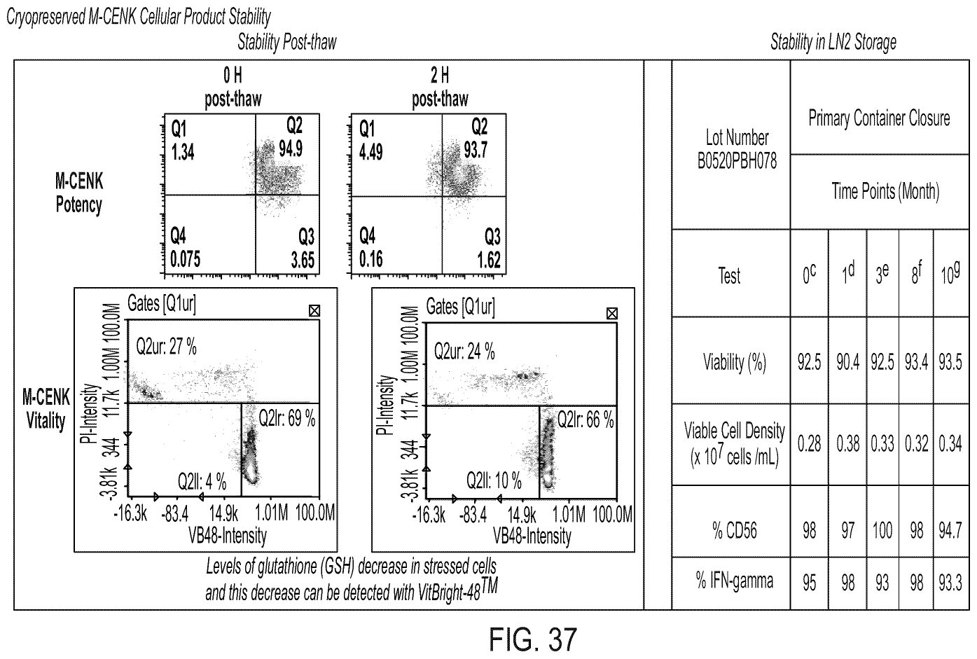

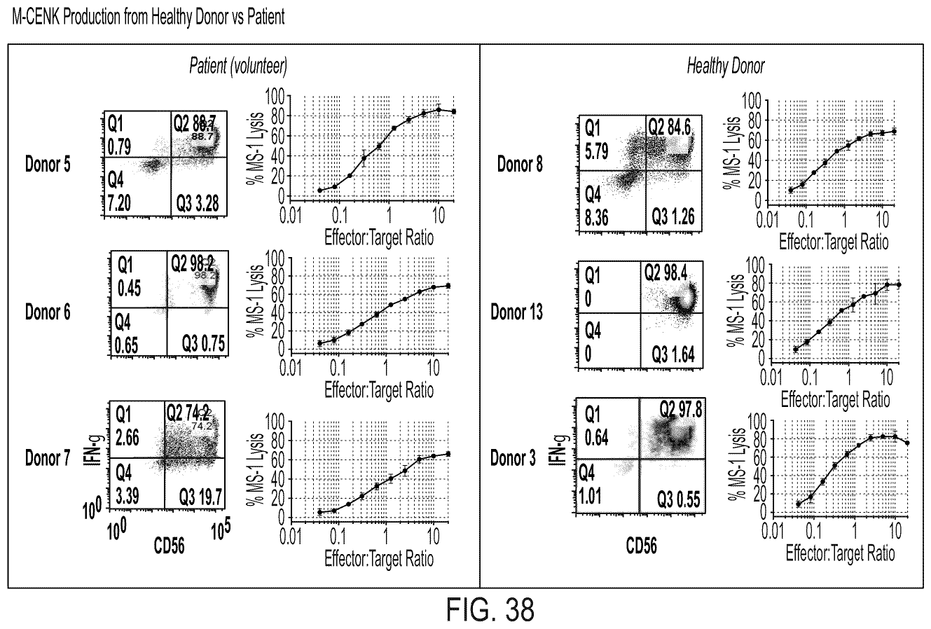

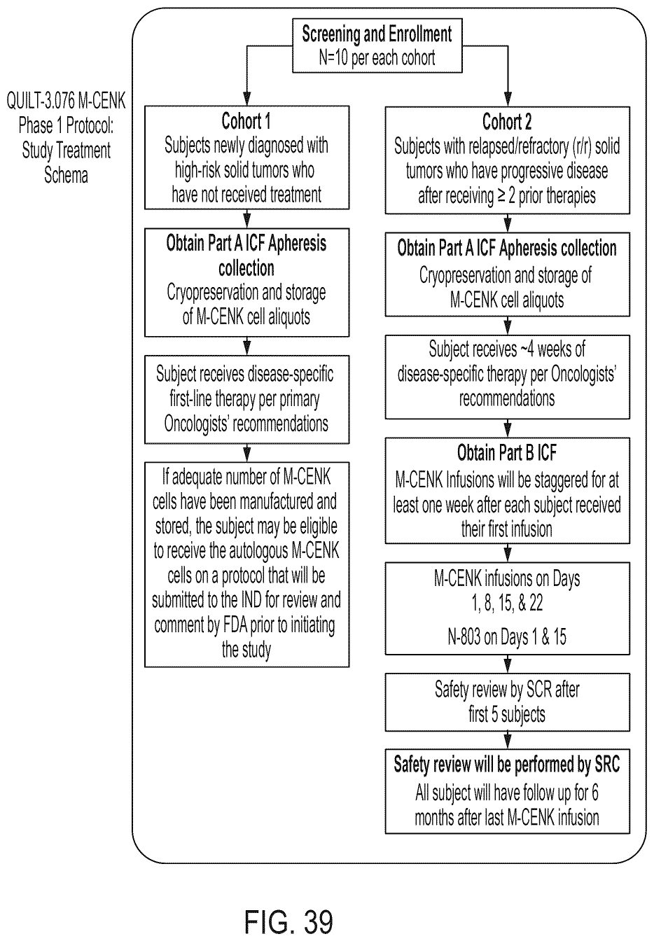

Apheresis Material Intermediate Stability in LN2 and Cryopreserved M-CENK Cellular Product Stability are shown in FIGS. 36-37 respectively. A comparison of M-CENK Production from Healthy Donor vs Patient (FIG. 38) shows that M-CENK may be obtained from both healthy donors and patients. FIG. 39 shows the Phase 1 protocol for clinical study QUILT-3.076, (Study of Autologous M-CENK in Subjects With Locally Advanced or Metastatic Solid Tumors)

Fusion Protein Scaffold 18/12/TxM Activates the IL-12, IL-15, and IL-18 Receptors to Induce Human Memory-Like Natural Killer Cells

In one embodiment, Fehniger and colleagues describe the creation of a novel triple-cytokine fusion molecule, 18/12/TxM, containing an IL-15 superagonist backbone (N-803) fused to IL-18 and IL-12. This trimeric molecule retained specific and unique IL-12, IL-15, and IL-18 activities, and generated potent human memory-like natural killer cells in vitro and in vivo. See Cubitt C C, et al, “A novel fusion protein scaffold 18/12/TxM activates the IL-12, IL-15, and IL-18 receptors to induce human memory-like natural killer cells”, Molecular Therapy: Oncolytics (2022), which is incorporated by reference in its entirety.

Natural killer (NK) cells are cytotoxic innate lymphoid cells that are emerging as a cellular immunotherapy for various malignancies. NK cells are particularly dependent on interleukin-15 (IL-15) for their survival, proliferation, and cytotoxic function. NK cells differentiate into memory-like cells with enhanced effector function after a brief activation with IL-12, IL-15, and IL-18. N-803 is an IL-15 superagonist comprised of an IL-15 mutant (IL-15N72D) bound to the sushi domain of IL-15Rα fused to the Fc region of IgG1, which results in physiological transpresentation of IL-15. Here, we describe the engineering of a novel triple-cytokine fusion molecule, 18/12/TxM, using the N-803 scaffold fused to IL-18 via the IL-15N72D domain and linked to a heteromeric single-chain IL-12 p70 by the sushi domain of the IL-15Rα. This molecule displays trispecific cytokine activity through its binding and signaling through the individual cytokine receptors. Compared to activation with the individual cytokines, 18/12/TxM induces similar short-term activation and memory-like differentiation of NK cells on both the transcriptional and protein level, and identical in vitro and in vivo anti-tumor activity. Thus, N-803 can be modified as a functional scaffold for the creation of cytokine immunotherapies with multiple receptor specificities to activate NK cells for adoptive cellular therapy.

Natural killer (NK) cells are cytotoxic innate lymphoid cells that make up approximately 5-20% of circulating blood lymphocytes and are important in the elimination of virally infected and malignantly transformed cells. NK cell function is tightly regulated by a balance of germ-line encoded activating, co-stimulatory, and inhibitory receptors expressed at the cell surface. Through these receptors, NK cells are able to recognize and spontaneously kill cells through the loss of self-identifying molecules such as major histocompatibility complex (MHC) class I that bind to inhibitory receptors on NK cells (detection of ‘missing self’) or by upregulating ligands recognized by activating receptors on NK cells that can overcome inhibitory signals. Human NK cells are identified by surface expression of CD56 and the absence of CD3 and can be categorized based on relative CD56 expression into the distinct CD56^{bright} and CD56^{dim} subsets, where CD56^{dim} NK cells typically express the FcγRIII (CD16), while CD56^{bright} NK cells have low or no expression.

NK cells constitutively express a number of cytokine receptors, and are particularly dependent on IL-15 for development, homeostasis, and function. IL-15 signaling has been shown to promote the survival, proliferation, and priming (at higher doses) of CD56^{bright} NK cells, and to enhance the cytotoxicity of the CD56^{dim} subset. There are three receptor subunits for IL-15 receptor forms: IL-15Rα (CD25), IL-15Rβ (CD122), and IL-15R (CD132). The signaling components of the IL-15 receptor are not private, with its β subunit shared with IL-2 and its γ subunit (common γ chain) with IL-2, IL-4, IL-7, IL-9, and IL-21. Physiologically, IL-15 mediates its effects through trans-presentation, whereby the high affinity IL-15Rα is expressed on the surface of accessory cells (such as dendritic cells and monocytes/macrophages) that present IL-15 to NK cells bearing the IL-15Rβγ_{c}. In addition to the effects mediated by IL-15, the cytokines IL-12 and IL-18 are also important for NK cell survival and function. The primary effect of IL-12 on NK cells occurs via STAT4-mediated signaling and include interferon-γ (IFN-γ) and tumor necrosis factor (TNF) production. IL-18 transduces signals that lead to MAPK and NF-kB activation and has been described to function synergistically with IL-12 and IL-15, while also priming NK cells for IFN-γ production. Indeed, paradigm-shifting studies have demonstrated that combined activation with IL-12, IL-15, and IL-18 induces a memory-like NK cells defined by enhanced proliferation, expression of the high-affinity IL-2 receptor αβγ (IL-2Rαβγ) and increased IFN-γ production after restimulation with cytokines, tumors, or via activating receptors. These cytokine-induced memory-like (ML) NK cells represent a promising NK cell therapy and have shown encouraging results in first-in-human clinical trials for relapsed/refractory AML patients.

N-803 is an IL-15 superagonist comprised of an IL-15 mutant (IL-15N72D) bound to the N-terminal structural (sushi) domain of IL-15Rα fused to the Fc region of IgG1. This results in accessory cell-independent trans-presentation of IL-15, prolonged in vivo pharmacokinetics, increased in vivo biological activity, and increased effector functions compared to IL-15. Given the potent functional effects induced by the combined stimulation of IL-12, IL-15, and IL-18, we hypothesized that the construction of a single molecule that could signal through all three cytokine pathways would be beneficial for generating memory-like NK cells for both research and clinical applications. As such, we constructed a fusion protein, 18/12/TxM, employing N-803 as a scaffold, linking IL-18 to the IL-15N72D domain and a heteromeric single-chain IL-12 p70 to the sushi domain of IL-15Rα, which is already linked to the Fc domain of human IgG1. This non-covalently associated, heterodimeric homodimer, triple-cytokine fusion protein retained specific and unique IL-18, IL-12, and IL-15 activities in vitro and in vivo.

The stability of the N-803 structure provides a biochemical strategy to decorate the ‘backbone’ with additional components, while maintaining IL-15 based signals. Here we investigated the ability of the novel 18/12/TxM fusion protein to generate memory-like NK cells, compared to the combination of individual recombinant human IL-12, IL-15, and IL-18. This included in vitro examination of individual activity of each cytokine receptor signaling, short term effector function, and ability to generate memory-like NK cells.

The Fusion 18/12/TxM Superkine Induces Signaling Via all Targeted Cytokine Receptors

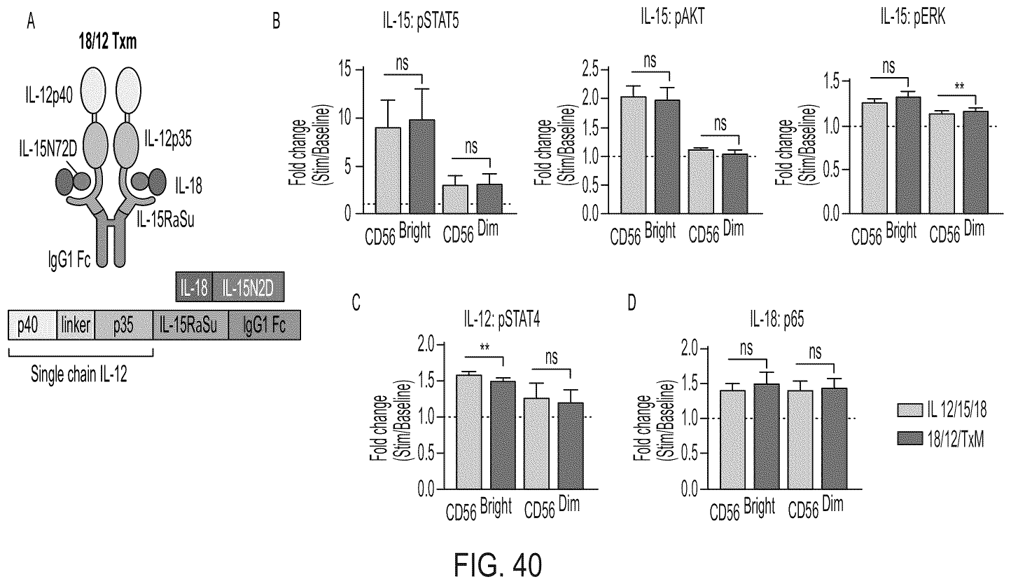

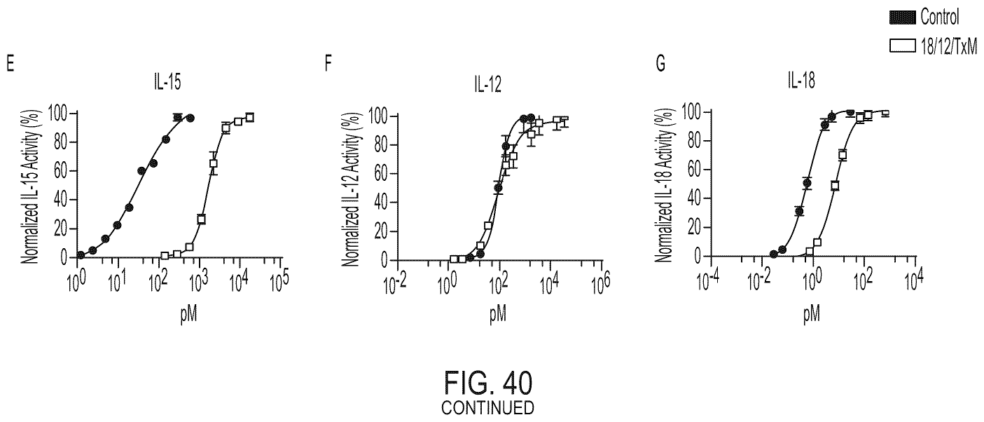

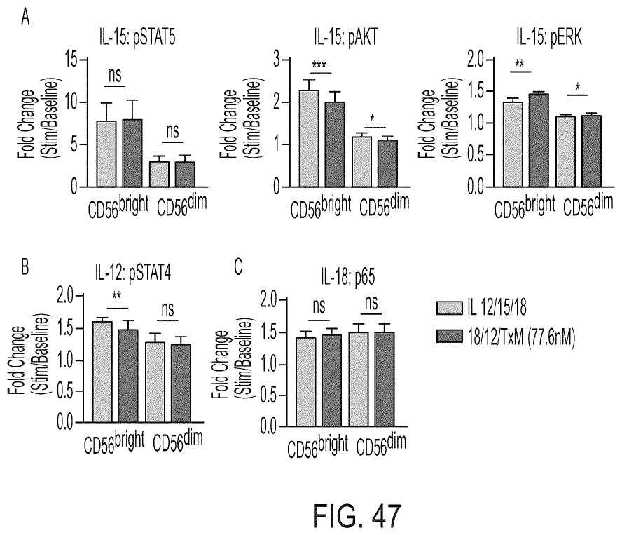

We first sought to construct a fusion protein consisting of human IL-18, IL-12, and IL-15 to replace individual use of the recombinant cytokines. To this end, N-803 (an IL-15 superagonist formerly known as ALT-803) was linked to IL-18 via the IL-15N72D domain and linked to heteromeric single-chain IL-12 p70 by the sushi domain of the IL-15Rα connected to the Fc domain of human IgG1 (FIG. 40A). The ability of this triple-cytokine fusion protein (henceforth referred to as 18/12/TxM) to induce signaling via all targeted cytokine receptors was assessed. Freshly isolated and purified NK cells were stimulated with 18/12/TxM (38.8 nM) or the optimal combination of recombinant human(rh) IL-12 (long/mL), IL-18 (50 ng/mL) and IL-15 (50 ng/mL) (IL12/15/18) and assessed for their ability to induce phosphorylation of key signaling intermediates at various time points. 18/12TxM induced IL-15 signaling through phosphorylation of STAT5, AKT, and ERK with similar efficiency to IL12/15/18 stimulation in both CD56_{bright} and CD56^{dim} NK cell subsets (FIG. 40B). At higher concentrations, 18/12/TxM (77.6 nM) induced slightly higher phosphorylated (p) ERK in CD56^{dim} cells, and slightly lower pAKT in CD56^{bright} NK cells (FIG. 47A). Phosphorylation of STAT4 by IL-12 signaling displayed modest but statistically significant difference at the lower TxM dose (38.8 nM) in CD56^{bright} NK cells (p=0.005), with no difference observed at the higher TxM concentration (77.6 nM) or in CD56^{dim} NK cells (FIG. 40C, FIG. 47B). Phosphorylation of p65 via IL-18 signaling was similarly induced by 18/12/TxM and IL-12/15/18 (FIG. 40D, FIG. 47C). Next, the ability of 18/12/TxM to activate individual cytokine bioassays was assessed. To elucidate the IL-15 activity, proliferation of a mouse hematopoietic cell line, the IL-2/15-dependent 32D-IL2/15Rβ (32Dβ) cells, was assessed. Increasing concentrations of 18/12/TxM or N-803 were added to 32Dβ cells and incubated for 3 days at 37° C., and proliferation was measured using the PrestoBlue cell viability reagent. The ability of 18/12/TxM to promote cell proliferation was reduced compared to that of N-803 (EC₅₀ of 1.7 nM vs 0.03 nM for N-803) possibly due to the linkage of IL-18 to the IL-15N72D domain (FIG. 40E). To determine the IL-12 activity of 18/12/TxM, activation of the IL-12 reporter HEK-blue (HEK12) cells, which express a STAT4-inducible secreted embryonic alkaline phosphatase (SEAP) gene, was assessed. Increasing concentrations of 18/12/TxM or recombinant IL-12 were added to HEK12 cells for 20-22 hours at 37° C. The activity of SEAP was measured using QUANTI-Blue (Invivogen), and the half-maximal effective concentration (EC₅₀) of IL-12 bioactivity was determined based on the relationship between absorbance and protein concentration, and the bioactivity of recombinant IL-12 was used as a positive control. The EC₅₀ of 18/12/TxM was 99.1 pM and 86.1 pM for rhlL-12, which demonstrates similar bioactivity to recombinant IL-12 (FIG. 40F). Finally, for IL-18, IL-18 reporter HEK-Blue (HEK18) cells, which express an NF-κB/AP-1-inducible SEAP gene, were plated with increasing concentrations of 18/12/TxM. After incubation for 20-22 hours at 37° C., the activity of SEAP was measured as described above. The EC₅₀ of 18/12/TxM was 7.1 pM, which was about 13-fold reduced compared to recombinant IL-18 (EC₅₀ of 0.54 pM), potentially due to the linkage of IL-18 to IL15N72D (FIG. 40G). Collectively, these data support that 18/12/TxM at adequate concentrations stimulates signals via the IL-12, IL-15, and IL-18 receptors.

Short Term Activation with 18/12/TxM Superkine Results in NK Cell Activation

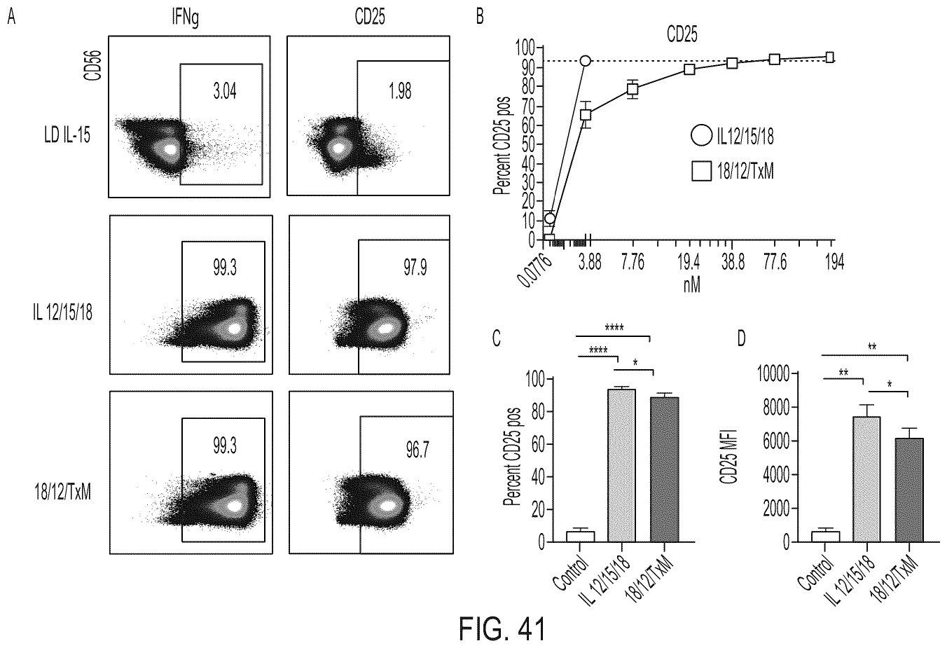

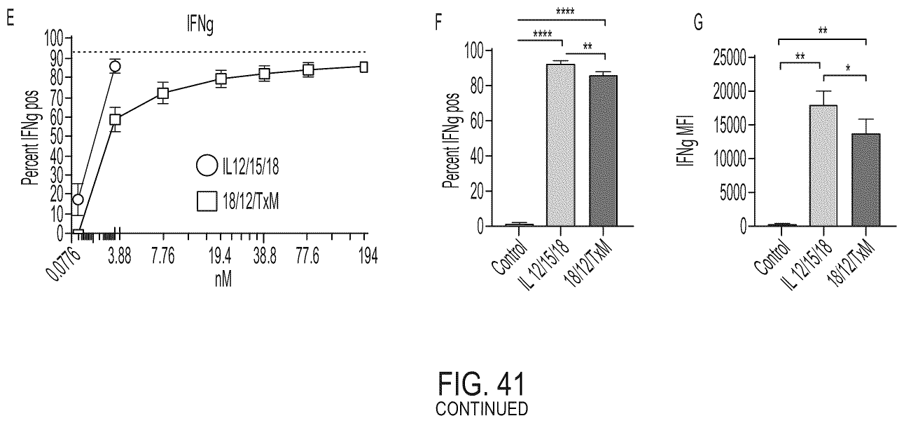

Short-term activation of human NK cells with IL-12, IL-15, and IL-18 leads to increased expression of the IL-2 receptor α (IL-2Rα, CD25) and enhanced production of IFN-γ. To evaluate the optimal concentration for 18/12/TxM activation, purified human NK cells were activated for 16 hours with increasing concentrations of 18/12/TxM or IL12/15/18. Induction of an activated phenotype was assessed as increased cell surface CD25 expression and intracellular IFN-γ, as compared to control, resting NK cells, as determined by flow cytometry (FIG. 41A). The optimal concentration for maximal induction of CD25 was reached at 38.8 nM 18/12/TxM, with an EC50 of 2.095 nM (FIG. 41B). Short term activation of purified human NK cells with 18/12/TxM at 38.8 nM or IL12/15/18 demonstrated similar induction of CD25 over control NK cells (FIG. 41C,D). Similarly, near maximal induction of IFN-γ was reached at 38.8 nM, with an EC₅₀ of 2.64 nM (FIG. 41E). Short term activation demonstrated similar induction of intracellular IFN-γ with 18/12/TxM compared to IL12/15/18, although both were significantly higher than the low-dose IL-15 (1 ng/mL) control (FIG. 41F,G). Collectively, these data show that the 18/12/TxM fusion protein results in nearly identical short-term activation via the IL-12, 15, and 18 receptors resulting in IFN-γ and CD25 expression, compared to the combination of rhIL-12, IL-15, and IL-18.

Activation with 18/12/TxM Superkine Stimulates NK Cell Proliferation

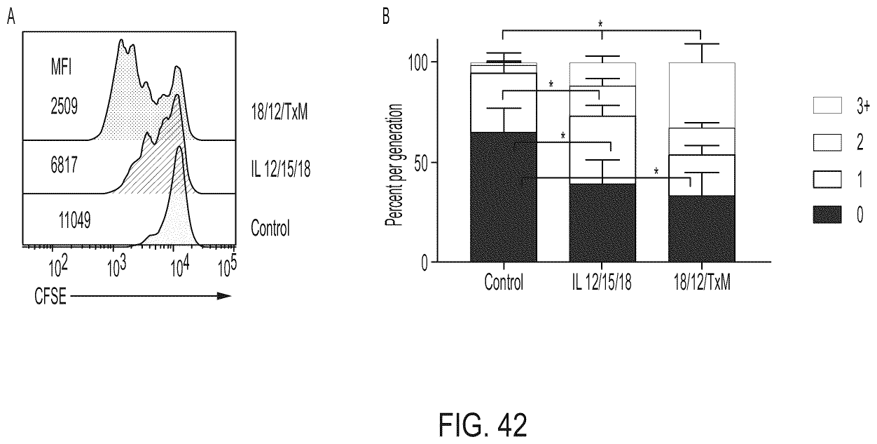

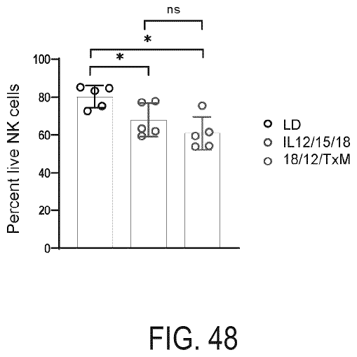

Previous studies have demonstrated that activation with IL-12, IL-15, and IL-18 leads to a memory-like phenotype that includes robust proliferation and expansion of NK cells. To address the ability of 18/12/TxM to induce proliferation, purified human NK cells were labeled with Carboxyfluorescein succinimidyl ester (CF SE), activated for 16 hours with 18/12/TxM (38.8 nM), IL12/15/18, or low-dose IL-15 (LD IL15). After activation, NK cells (including both CD56^{dim} and CD56^{bright} subsets) were washed and rested in LD IL15 for 6 days. In agreement with previous data, activation with IL-12/15/18 or 18/12/TxM induced robust proliferation, as compared to those activated with low-dose IL-15 (FIG. 42A,B). Interestingly, activation with 18/12/TxM in this set of experiments resulted in enhanced proliferation compared to IL12/15/18, with an increased proportion of NK cells expanding beyond 3 generations (FIG. 42B). This increased cell cycling with 18/12/TxM was not attributed to differences in viability between the activating conditions (FIG. 48). This enhanced proliferation may be attributed to the N-803 scaffold, which induces proliferation to a greater extent than IL-15 alone due to enhanced signaling from the IL-15Rα and IgG1-Fc components, or alternatively related to concurrent signals via the IL-12, IL-15, and IL-18 receptors.

Multidimensional Phenotypic Changes are Similar Between IL12/15/18 and 18/12/TxM Induced Memory-Like NK Cells

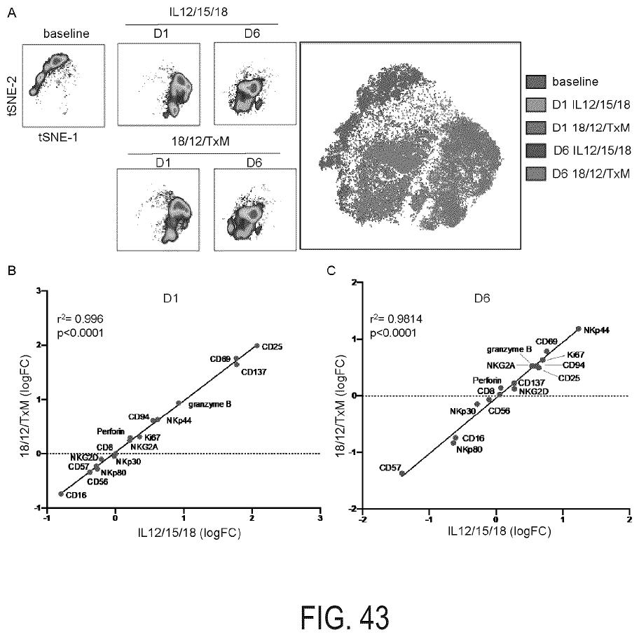

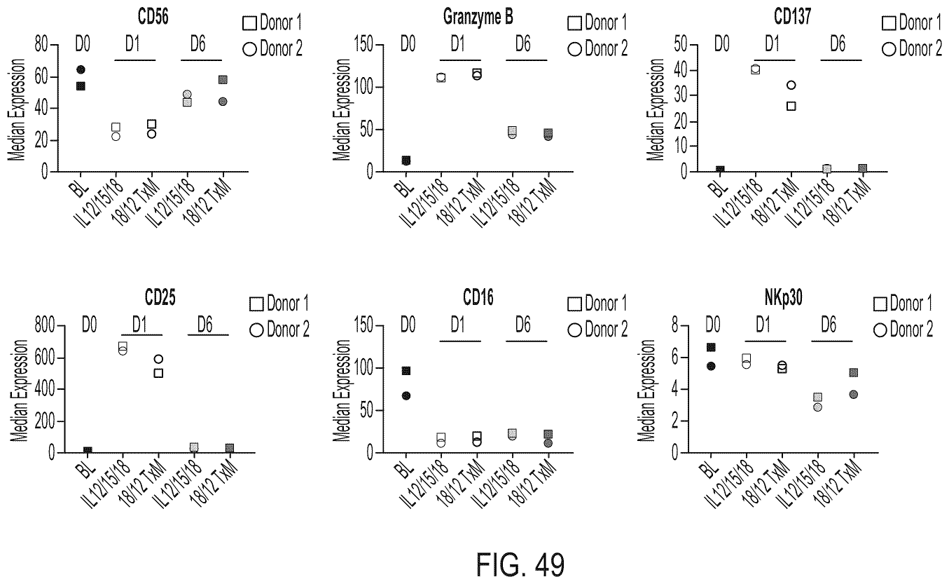

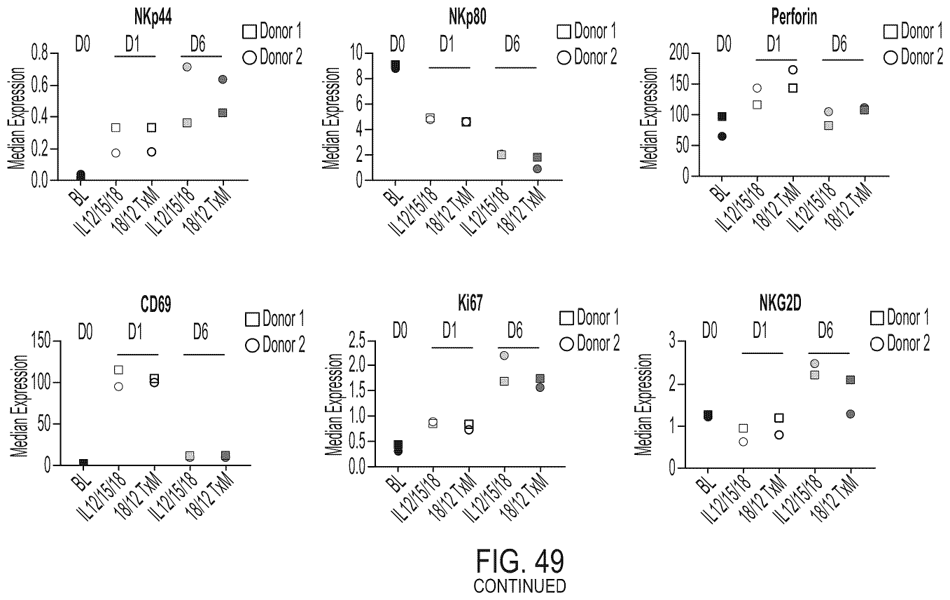

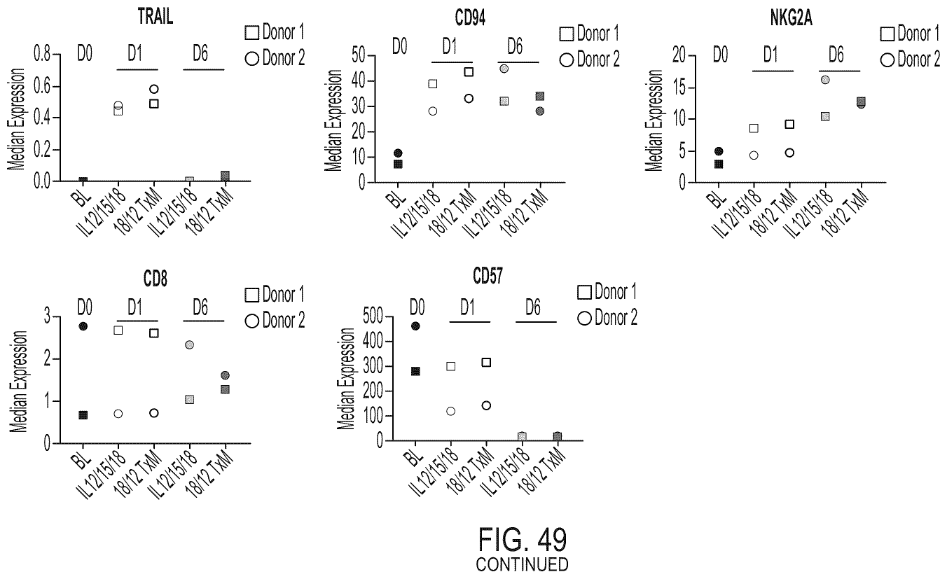

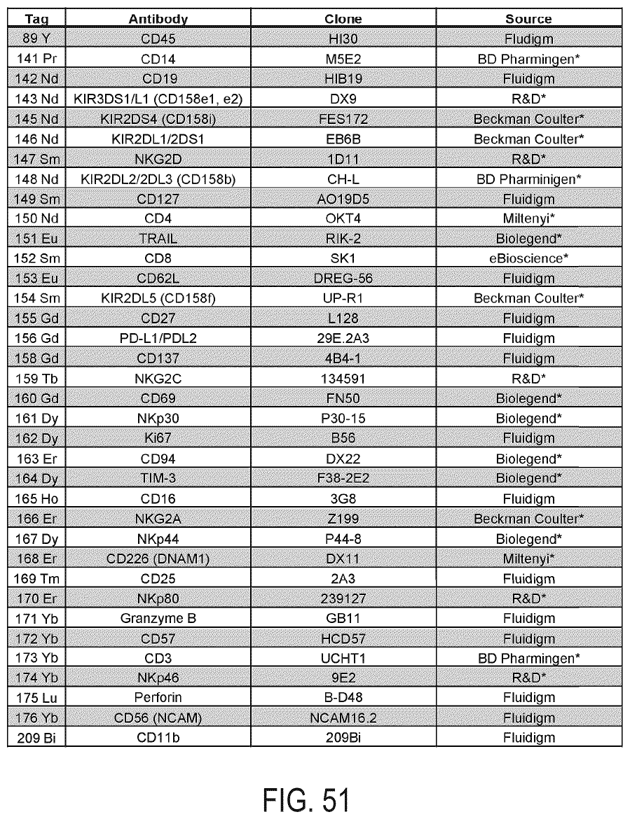

Memory-like NK cells undergo dramatic changes in a large number of cell surface and intracellular markers both immediately after activation with IL12/15/18 and 6 days after differentiation. A custom mass cytometry panel was previously developed including markers for NK cells lineage, maturation, and functional capacity (FIG. 51), and identified a ML NK cell multidimensional phenotype. To compare the multidimensional phenotypes, human NK cells were profiled using mass cytometry before activation (baseline), after 16 hours of incubation with IL-12/15/18 or 18/12/TxM (D1) and six days post-activation to allow time for ML differentiation (D6). Using the median expression of markers, tSNE analysis revealed distinct NK cell populations for baseline, Day 1, and Day 6 after activation. Notably, the same particular clustering NK cell subsets was identified when activated with either IL12/15/18 or 18/12/TxM (FIG. 43A). Furthermore, comparison of the changes in median expression after overnight activation of well-defined markers of acute NK cell activation such as increased CD25, CD69, and CD137 and decreased CD56 and CD16 were identical between IL12/15/18 and 18/12/TxM activated NK cells (FIGS. 43B, 49). In accordance with previous studies of differentiated memory-like NK cells, both IL12/15/18 and 18/12/TxM activated NK cells demonstrated similar upregulation of NKG2A, CD69, Ki67, CD25, CD137, granzyme B, perforin, and the activating receptors NKp44, NKG2D, and CD94 at Day 6. They also demonstrated similar downregulation of CD56, CD16, CD57, NKp30, and NKp80, as has been previously reported (FIG. 43C). Using mass cytometry, we were able to determine that the trimeric superkine 18/12/TxM was able to induce a memory-like NK cell phenotype that is identical to the induction from the individual recombinant cytokines combined. These data indicate that 18/12/TxM activation results in short- and long-term changes in NK cells, similar to IL-12, IL-15, and IL-18 activation.

18/12/TxM Induces Functional Memory-Like NK Cells In Vitro

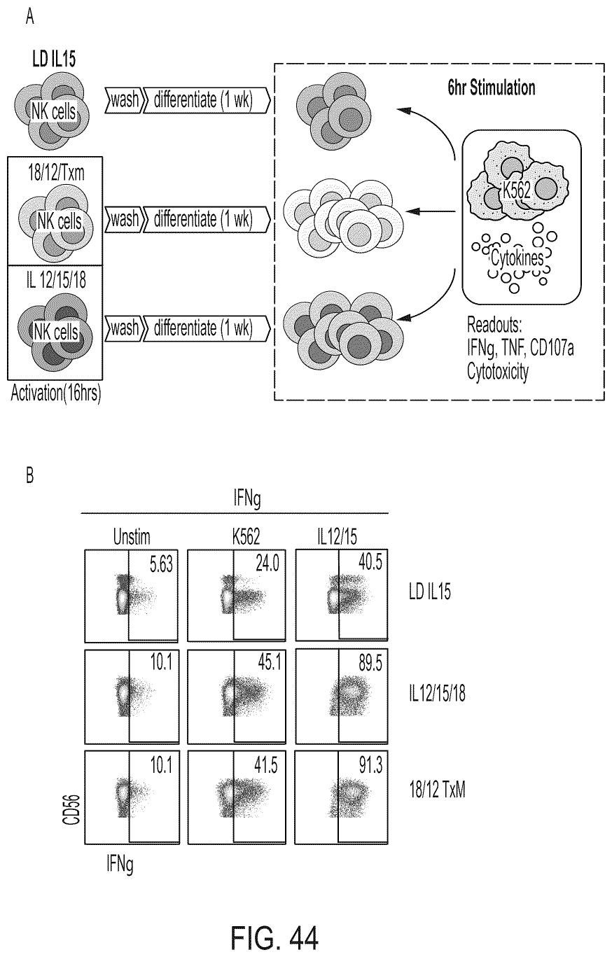

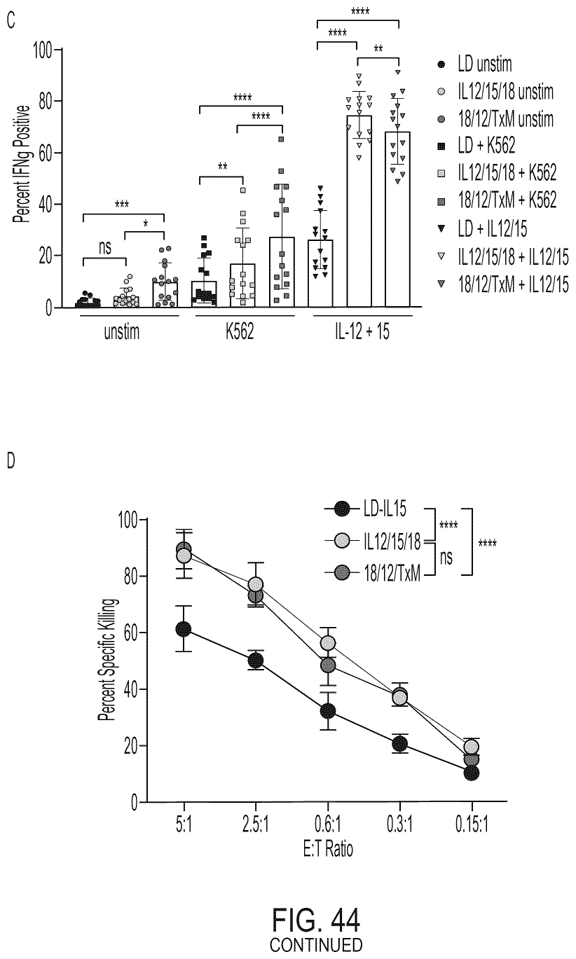

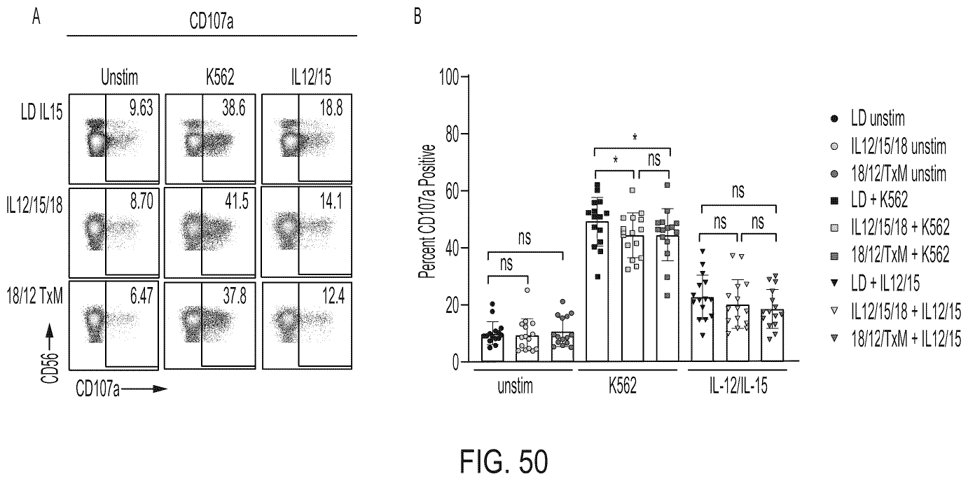

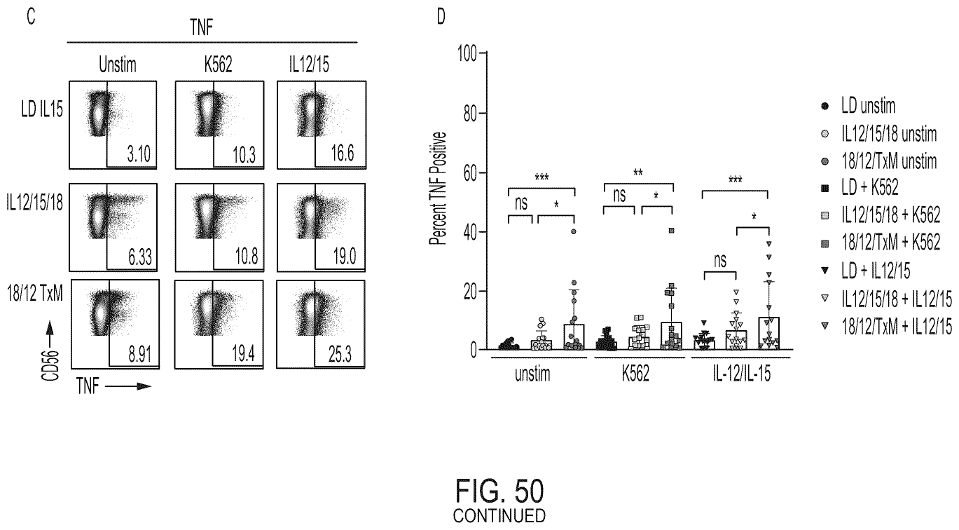

Previous studies have shown that ML NK cells can be induced ex vivo following overnight stimulation of purified NK cells with saturating amounts of IL12/15/18. These cells exhibit ML properties such as 1) enhanced proliferation, 2) expression of IL-2Rα, 3) increased IFN-γ production, and 4) augmented cytotoxicity mediated by perforin and granzymes. To demonstrate generation of ML NK cells by 18/12/TxM, primary human NK cells were activated with 18/12/TxM (38.8 nM), IL12/15/18 or LD IL15 for 16 hours, washed, and supported in low-dose IL-15 for 6 days to allow memory-like differentiation (FIG. 44A). The production of IFN-γ as a functional readout for the generation of ML NK cells was assessed following 6-hour re-stimulation with cytokines (IL-12 [10 ng/ml] and IL-15 [50 ng/mL]) or leukemia targets (K562 cells, at 5:1 effector:target ratio) (FIG. 44B). Activation with 18/12/TxM induced IFN-γ expression to a slightly greater extent than IL-12/15/18 after K562 stimulation (FIG. 44C). Expression of IFN-γ after IL-12+IL-15 stimulation was slightly higher in IL12/15/18 activated NK cells, compared to 18/12/TxM, but was robustly induced over the LD control in both conditions. (FIG. 44C). Induction of CD107a (a surrogate marker for degranulation) between LD, IL12/15/18, and 18/12/TxM after K562 stimulation was similar, which is consistent with previous reports that degranulation is not affected by memory-like differentiation (FIG. 50A-C). Interestingly, activation with 18/12/TxM resulted in higher expression of TNF even without stimulation, suggesting that 18/12/TxM is inducing higher baseline expression of this cytokine (FIG. 50D-F). In addition to cytokine secretion, the ability of 18/12/TxM to promote tumor killing was assessed in a standard 4-hour cytotoxicity assay using K562 target cells. Specific killing of target cells was identical between IL12/15/18 and 18/12/TxM activated NK cells, and greater than LD NK cells at all E:T ratios evaluated (FIG. 44D). These data indicate that that the 18/12/TxM molecule was capable of inducing memory-like functions, including enhanced IFN-γ and cytotoxicity, to the same extent as IL12/15/18.

18/12/TxM Activation Induces a Molecular Program Similar to IL-12, IL-15, and IL-18

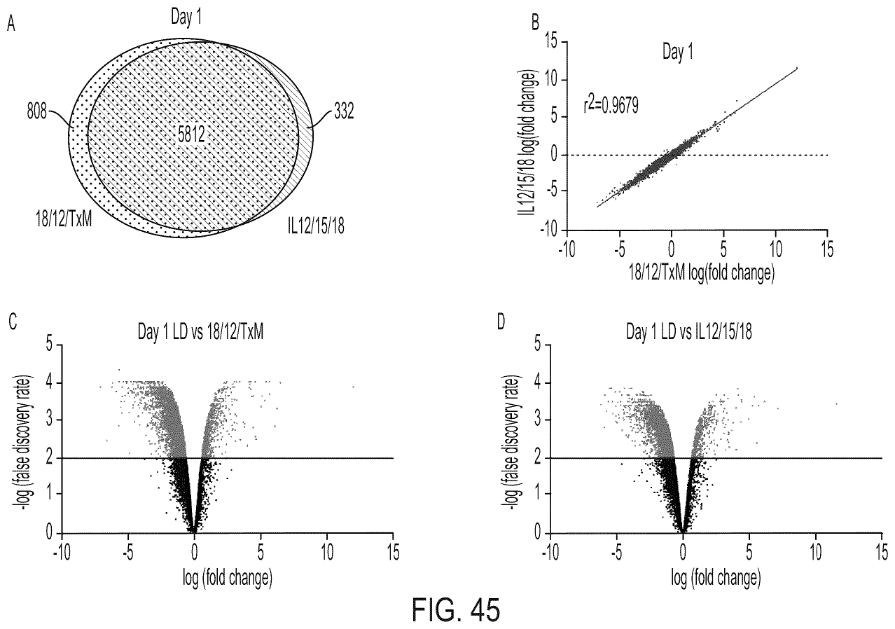

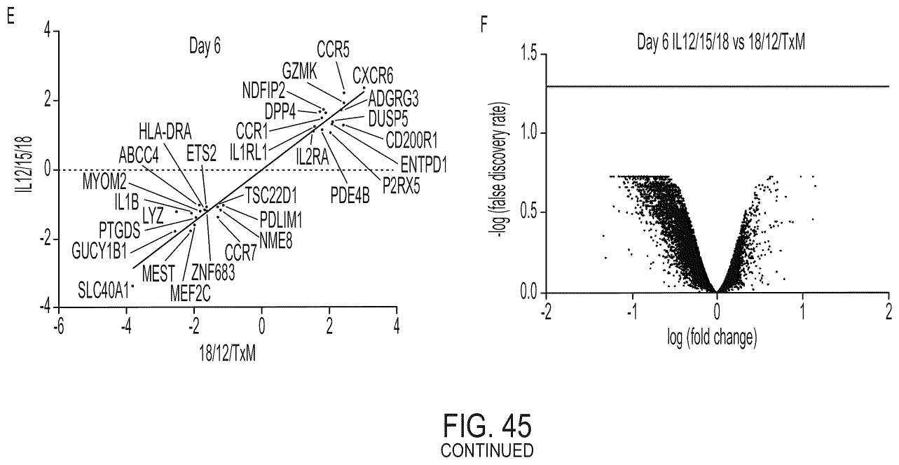

To complement the phenotypic and functional similarities induced by 18/12/TxM and IL12/15/18, we performed bulk RNA sequencing. Purified NK cells from three different donors were isolated, and RNA was isolated before (baseline), after overnight activation (D1), and six days supported by IL-15 (D6) after activation with either 18/12/TxM, IL12/15/18, or IL-15. Analysis of transcript counts revealed a similar gene expression profile on D1 after activation between IL12/15/18 and 18/12/TxM activated NK cells, when contrasted to LD IL-15. Analysis of the genes that were significantly differentially expressed (p<0.05) on D1 following IL12/15/18 or 18/12/TxM stimulation demonstrated that the vast majority (5,812 genes) of changes were shared after either treatment, with 808 unique genes expressed in the TxM condition, and 332 unique genes following IL12/15/18 treatment (FIG. 45A). Indeed, when directly comparing genes expressed in NK cells activated with 18/12/TxM or IL12/15/18, their expression profiles were nearly identical (r²=0.9679) one day after activation (FIG. 45B). Consistent with phenotypic observations on D1, brief activation with both 18/12/TxM and IL12/15/18 led to a dramatic increase in expression of IL2Rα (CD25), IFN-γ, granzyme B, LTA, TNFSF4 (OX40L), NIFK, CCL3 and CSF2 (GM-CSF) (FIG. 45C,D). On Day 6 of differentiation after activation supported by LD IL-15, there were no statistically significant differentially expressed genes between LD IL-15 and 18/12/TxM or IL12/15/18 activated NK cells. While the vast majority of gene expression changes were minimal (logFC <0.5 or >−0.5), a direct comparison of genes induced in 18/12/TxM and IL12/15/18 activated NK cells on day 6 that trended differently compared to LD (logFC >1 or <−1) revealed similar changes between the two treatment groups (FIG. 45E). Despite the dramatic molecular activation profile one day after activation, by day 6 there were no statistically significant genes that were differentially expressed between LD IL-15 and IL12/15/18 activated NK cells. This is consistent with previous work done in our laboratory and is likely due to the heterogeneity of NK cells at day 6 that underwent memory-like differentiation, and the overwhelming IL-15-induced transcriptional profile that may mask a unique transcriptional profile. Some differentially expressed genes trended differently between LD IL-15 and 18/12/TxM or IL12/15/18 treated NK cells at Day 6, including increased expression of CXCR6, CCR1, and Granzyme K. A direct comparison of the gene expression changes induced by 18/12/TxM or IL12/15/18 showed no statistically significant differences, suggesting that they are inducing similar transcriptional profiles by Day 6 (FIG. 45F). Thus, using bulk RNA sequencing approaches on enriched NK cells, 18/12/TxM and IL12/15/18 induce nearly identical transcriptional changes after 24 hours, and were both distinct from control NK cells. However, this analysis approach does not identify significant differences between day 6 ML NK cells induced with either initial activation. Based on the subsets of ML NK cells with enhanced function, we expect that only a proportion of cells at day 6 represent functional ML NK cells with unique transcriptional signatures. In this setting, single-cell RNAseq approaches would be required to identify subset-based transcriptome changes.

18/12/TxM Induces Memory-Like NK Cell Anti-Tumor Activity In Vivo

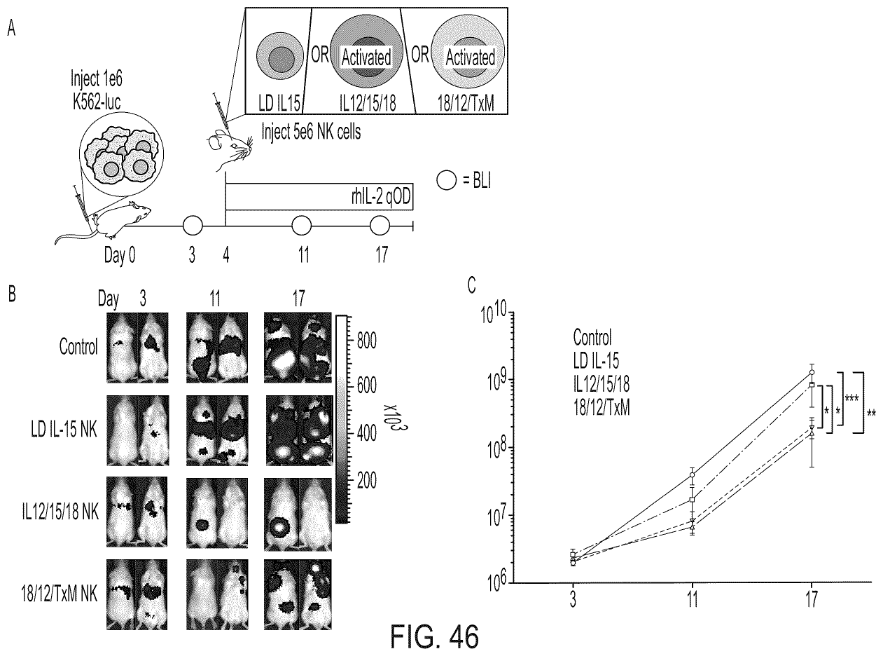

To confirm that the molecular and phenotypic changes induced in vitro by 18/12/TxM would also translate to enhanced in vivo functionality, we compared the anti-tumor activity in NOD-SCID-IL2Rg^{-/- (NSG) mice engrafted with leukemia. Briefly, NSG mice were engrafted with luciferase-expressing K}562 tumor cells (0.5×10⁶ cells/mouse) and injected 4 days later with NK cells (3-5×10⁶ cells/mouse) that had been pre-activated with 18/12/TxM, IL-2/15/18, or LD IL-15 (FIG. 46A). Tumor growth was assessed using whole body bioluminescence imaging (BLI) on day 3, day 11, and day 17 (FIG. 46B). ML NK cells induced with either IL12/15/18 or with 18/12/TxM demonstrated enhanced tumor control at Day 17 (FIG. 46C). These data demonstrate that activation of NK cells with 18/12/TxM can induce a ML NK cell phenotype, similar to that induced by IL12/15/18, which exerts enhanced control of tumor targets in vivo.

The novel fusion protein 18/12/TxM was constructed using N-803 as a scaffold, linking IL-18 to the IL-15N72D domain and linking heteromeric single-chain IL-12 p70 to the sushi domain of IL-15Rα, which is already linked to the Fc domain of human IgG1. This non-covalently associated, heterodimeric homodimer, triple-cytokine fusion protein retained specific and unique IL-18, IL-12, and IL-15 activities in vitro, as measured by activation, proliferation, and signaling through cognate receptors. Furthermore, 18/12/TxM exhibited functions equivalent to the combination of the individual cytokines when used at the appropriate concentration on primary NK cells ex vivo after overnight stimulation and after 6 days of in vitro differentiation into memory-like NK cells and in vivo in NSG mice. The ability of 18/12/TxM to induce this memory-like phenotype was confirmed at the protein and transcriptional level using high-dimensional methods including mass cytometry phenotyping and bulk RNA-sequencing. These phenotypic changes translated to equivalent cytotoxic effector functions in vitro and similar tumor control in vivo. Thus, 18/12/TxM is an alternative to IL-12, IL-15 and IL-18 for the generation of memory-like NK cells.

Interestingly, activation with 18/12/TxM resulted in greater levels of proliferation in purified NK cells than the individual cytokines combined. This finding suggests that there may be distinct biological outcomes resulting from simultaneous engagement of the IL-12, IL-15, and IL-18 receptors on the same cell rather than sequential activation of the receptors on the same or different cells. Another possibility is that the spatial linking of the cytokines on the scaffold that results in enhanced membrane clustering of cytokine receptors and signaling molecules as a result of binding from the trimeric molecule. It may also be possible that the Fc portion of 18/12/TxM is activating NK cells via downstream signaling events following engagement with the FcγRIII receptor (CD16). However, given the observation that the increased proliferation occurred in both CD56^{dim} (CD16+) and CD56^{bright} (CD16−) NK cells, it may also be possible that Fc-FcR interactions allow CD16+ NK cells to readily trans-present the 18/12/TxM to nearby NK cells. Further investigation using an FcR null variant of the 18/12/TxM would be required to clarify this potential contribution.

Minimal differences in gene expression were observed between LD IL15 and IL12/15/18-activated NK cells at Day 6. This is consistent with previous observations that only some NK cells are able to fully undergo memory-like differentiation, therefore making it difficult to identify their gene signatures with bulk RNA-sequencing methods and following extensive IL-15-supported culture in vitro. Further studies using more in-depth sequencing methods such as single-cell RNA-sequencing will be essential for characterizing the unique transcriptional changes in memory-like differentiated NK cells. It is also possible that memory-like differentiation is orchestrated predominantly by epigenetic changes, which can be clarified with methods such as ATAC-seq.

IL-15 has been used as an ideal candidate for clinical immunotherapy combinations due to its ability to stimulate NK cell (and CD8+ T cell) activation. However, physiological activation with IL-15 requires binding to the IL-15Rα-chain prior to activating target cells, which limits the research and clinical roles of free IL-15. N-803, consists of human IgG1 Fc fused to two IL-15Rα subunits bound to an IL-15 superagonist (N72D mutation that enhances biological activity), resulting in higher biological activity and longer serum half-life compared with free IL-15. Previous studies have demonstrated that preactivation of NK cells with IL12/15/18 results in ML NK cell differentiation that represents a promising approach to enhancing adoptive allogenic NK cell therapy. However, the use of these individual cytokines alone or in combination for research and clinical purposes can be subject to production issues and lot variability. Additionally, this superkine presents a promising platform for exchanging out different NK cell activating cytokines (IL-2, IL-21) or tumor targeting molecules (e.g., CD20, EGFR, HER2 or CD34) to direct activated NK cells to kill tumor cells. Indeed, N-803 has been used as a functional scaffold fused with CD20 targeting antibody components and demonstrated superior anti-tumor activity than the individual components alone. Other studies have demonstrated enhanced anti-tumor activity when N-803 is used in combination with either tumor targeting or checkpoint inhibitory antibodies, which represents a promising avenue for the development of additional fusion proteins.

In this disclosure, it is demonstrated that the fusion of three distinct cytokines via a human IgG1Fc connection induced equivalent activity to the individual cytokines combined, in vitro and in vivo. While these studies used 18/12/TxM to pre-activate NK cells in vitro before infusion, the Fc backbone confers additional in vivo half-life that could support its use in vivo in other contexts. Additionally, the use of the N-803 protein scaffold linked to three distinct cytokine targets represents a novel method to expand and stimulate NK cells for adoptive cell therapy.

Materials and Methods

Recombinant Proteins: hIL18/IL12/TxM protein, lot #305-86(1) and N-803 protein, lot #01062016 were manufactured and purified at Altor BioScience, Miramar, FL. Endotoxin-free, recombinant human (rh)IL-12 (Biolegend), IL-15 (Miltenyi), IL-18 (InVivoGen) and IL-2 (R&D Systems, Minneapolis, MN) were used in these studies.

Flow cytometry antibodies: The following Beckman Coulter antibodies were used: CD3 (clone UCHT1, CD45 (clone A96416), CD56 (clone N901), NKG2A (clone Z199.1), NKp46 (clone BAB281). The following BD antibodies were used: CD16 (clone 3G8), IFN-□(clone B27), CD107a (clone H4A3), CD57 (NK-1) CD69 (FN50), CD137 (clone 4-1BB), Perforin (clone dG9), Ki67 (clone B56), ERK1/2 (pT202, pY204), AKT (pS473), STAT4 (38/p-Stat4), STAT5 (47/Stat5, pY694), p38 (pT290/pY182), p65 (pS529). The following Biolegend antibodies were used: NKG2D (clone 1D11), NKp30 (clone P30-15). NKp44 (clone P44-8), IgG1 control (clone MG1-45). The following eBioscience antibodies were used: Granzyme B (GB12), TNF (clone Mal) 11).

Cell lines: K562 cells (ATCC, CCL-243) were obtained from ATCC in 2008, viably cryopreserved, thawed for use in these studies, and maintained for no more than 2 months at a time in continuous culture as described. Prior to our studies, the K562 cells were authenticated by confirming cell growth morphology (lymphoblast), growth characteristics, and functionally as NK-cell—sensitive targets in 2014 and 2015. Cells were cultured in RPMI1640 supplemented with L-glutamine, HEPES, NEAA, sodium pyruvate, and Pen/Strep/Glutamine containing 10% FBS (Hyclone/GE Healthcare, Logan, UT).

HEK-BlueIL-18 cells (Interleukin-18 sensor cells) and HEK-Blue IL-12 cells (Interleukin-12 sensor cells) from Invivogen (San Diego, CA) were cultured in complete HEK-Blue media (I10 media) consisting of IMDM, 10% FBS (HyClone/GE Healthcare, Logan, UT); 1× penicillin-streptomycin-glutamine (Thermo Fisher Scientific, Dallas, TX); 100 ug/ml normacin, and 1X HEK-Blue selection (InvivoGen, San Diego, CA). 32D-IL2/15Rβ (32D13) cells were constructed at Altor BioScience (Miramar, FL), cultured in complete 32Dβ media containing IMDM-10 media plus 25 ng/ml rh IL-2, and maintained at a cell density between 1.5×10⁴-2×10⁶ cells/ml at 37° C. and 5% CO₂.

NK-cell purification and cell culture: Human platelet apheresis donor PBMCs were obtained by ficoll centrifugation. NK cells were purified using RosetteSep (StemCell Technologies, ≥95% CD56⁺CD3⁻) and used for selected experiments. Cells were plated at 3-5×10⁶ cells/mL and preactivated for 16 hours using 38.8 nM 18/12/TxM (9.5 ug/mL), rhIL-12 (10 ng/mL)+rhIL-18 (50 ng/mL)+rhIL-15 (50 ng/mL) or control conditions (rhIL-15, 1 ng/mL). Cells were washed 3 times to remove cytokines, and cultured for 6 days in HAB10 complete media containing RPMI 1640 medium+10% human AB serum (Sigma-Aldrich, St. Louis, MO) supplemented with rhIL-15 (1 ng/mL) to support survival, with 50% of the medium being replaced every 2-3 days with fresh rhIL-15.

Assessment of IL-18 and IL12 activity: HEK-Blue IL-18 and HEK-Blue IL-12 cells were maintained in complete HEK-Blue Media at 37° C. and 5% CO₂. HEK-Blue Selection was added to the growth media after two passages, as per the manufacturers' cell handling recommendations. Growth media was renewed twice a week and cells were passaged when 70-80% confluency was reached. To measure activity of IL-18 or IL12, the respective sensor cells were detached in PBS and resuspended in Complete HEK-Blue assay media at 280,000 cells/ml. Twenty microliters of half-log serially diluted cytokine control and hIL18/IL12/TxM (in the concentration range described below) was added to a flat bottom 96 well plate, followed by addition of 180 uL of cells, for a final cell count of ˜50,000 cells in 200 uL. The plates were incubated for ˜20 hours at 37° C. and 5% CO₂. To assess the activity of IL-18 or IL-12, the resulting secreted alkaline phosphatase was quantified using QUANTI-Blue detection reagent (Invivogen, San Diego, CA). QUANTI-Blue reagent was prepared as per the manufacturers' instructions. After warming QUANTI-Blue to room temperature, 180 uL was added to 20 uL of culture supernatant in a 96 well flat bottom plate and incubated for 18 hours at 37° C. and 5% CO₂. Absorbance was then measured at 650 nm to determine cell activation based on reduction of QUANTI-Blue by secreted alkaline phosphatase. The EC₅₀ of IL-18 or IL-12 bioactivity of 18/12/TxM was determined from the dose response curve generated using non-linear regression variable slope curve fitting with GraphPad Prism 7.

Concentration Range: For detection of IL-18 activity, half-log serial dilution ranging from 10 ng/ml (556 pM) to 0.05 pg/ml (0.0028 pM) for IL-18 and 3350 ng/ml (13673 pM) to 0.0167 ng/ml (0.0683 pM) for 18/12/TxM was performed. This corresponds to final pM concentrations of 56 pM to 0.00028 pM for the IL-18 cytokine and 1367 pM to 0.00683 pM for 18/12/TxM. For detection of IL-12 activity, half-log serial dilutions ranging from 1000 ng/ml (17483 pM) to 5 pg/ml (0.0875 pM) for IL-12 and 85.7 ug/ml (349,796 pM) to 0.428 ng/ml (1.748 pM) for 18/12/TxM was performed. This corresponds to final pM concentrations of 1748.3 to 0.00875 pM for IL-12 cytokine and 34,979.6 to 0.1748 pM for 18/12/TxM molecule.

Assessment of IL-15 activity: To measure IL-15 activity, the assay plate was prepared as follows: 100 uL of IMDM-10 media was added to each well in a 96 well, flat bottom plate. Next 100 uL of 4× concentration of N-803 (225 ng/ml; ˜2400 pM) or IL18/IL12/TxM (18,000 ng/ml; ˜73468 pM) was added to column 1. The drugs were 2-fold serially diluted to column 10, leaving 100 uL in each well. The cells were washed 3× with IMDM-10 media, resuspended at a density 1×10⁵ cell/ml in IMDM-10 media, and 100 uL of the cells was added to the assay plate from column 1 to 11, for a total assay volume of 200 uL. One hundred uL of IMDM-10 was added to column 12. The assay plate was placed at 37° C. and 5% CO₂ for ˜72 hours. To assess the activity of IL-15, 20 uL of 10× PrestoBlue Cell Viability Reagent was added directly to the assay plate after ˜72 hours, and the plate is placed at 37° C. and 5% CO₂ for an additional ˜4 hours. Absorbance was measured at 570 nm and at 600 nM for normalization. Using column 11 (cells with no drug) as a negative control, the EC₅₀ of IL-15 bioactivity of hIL18/IL12/TxM was determined from the dose response curve generated using non-linear regression variable slope curve fitting with GraphPad Prism 7.

Phosphorylation assays: Freshly isolated human NK cells were incubated in HAB10 media without cytokines at 37° C. for 30 min. Individual cytokines (IL-12, IL-15, or IL-18) were added to wells at the indicated concentration for varying time intervals (2-hour stimulation for STAT4, 1 hour stimulation for Akt and ERK, and 15 minute stimulation for NF-□3-P65, STAT5, and P38 detection). After incubation, cells were fixed with 4% paraformaldehyde (PFA) and incubated at room temperature for 10 minutes. The cells were then pelleted and resuspended in cold 100% methanol and incubated and 4° C. for 30 minutes. Cells were washed 3 times with FACs buffer (PBS, 0.5% BSA, 2 mM EDTA). After washing, cells were suspended in surface antibody master mix (CD3, CD16, CD56, CD45) as well as the appropriate phosphoflow antibodies and stained overnight at 4° C. The next morning, cells were washed twice, and samples were acquired on a BeckmanCoulter Gallios flow cytometer and analyzed using FlowJo Version 9.3.2 (TreeStar) software.

Functional assays to assess cytokine production: Control and memory-like NK cells were harvested after a rest period of 6 days to allow memory-like NK cell differentiation to occur. Cells were then restimulated in a standard functional assay. Briefly, cells were incubated for 6 hours with K562 leukemia targets at an effector to target (E:T) ratio of 5:1 unless otherwise noted, in presence of CD107a. After 1 hour of stimulation, Brefeldin A and Monensin (GolgiStop/GolgiPlug, BD) were added, and 5 hours later the cells were stained for CD45, CD3, CD56, CD25. Cells were fixed (Cytofix/Cytoperm, BD) and permeabilized (Perm/Wash, BD) before the staining of intracellular IFN-□ and TNF. Cells were acquired on a Gallios 3 flow cytometer and analyzed using FlowJo Version 9.3.2 (TreeStar) software.

Assessment of Specific Killing: On D6 or D7 post-activation, control or ML-NK cells were re-suspended in 1 ng/mL IL-15 and challenged with K562 targets at various E:T ratios, in a standard four-hour 51Chromium release assay. 51Cr release was detected on a Wallac Microbeta Tri-lux Scintillation Counter. The percent specific lysis was calculated by: [(cpmₑₓₚ−cpmₛₚₒₙₜₐₙₑₒᵤₛ)/cpmₘₐₓ−cpmₛₚₒₙₜₐₙₑₒᵤₛ)]*100.

Flow cytometric analysis: Cell staining was performed as described previously, and data were acquired on a Gallios flow cytometer (Beckman Coulter, Indianapolis, IN) and analyzed using Kaluza Version 1.2 (Beckman Coulter) or FlowJo Version 9.3.2 (TreeStar) software. Statistical analysis were done using GraphPad Version 7.0 software.

RNA-sequencing: One million purified NK cells were frozen in Trizol at −80° C. until RNA isolation using the Direct-zol RNA MicroPrep kit (Zymo Research). NextGen RNA sequencing was performed using an Illumina Hi Seq 2500 sequencer. RNASeq reads were aligned to the Ensembl release 76 top-level assembly with STAR version 2.0.4b. Gene counts were derived from the number of uniquely aligned unambiguous reads by Subread: featureCount version 1.4.5. Analysis of sequencing data was performed using Phantasus, a browser-based gene expression analysis software. Differential expression analysis was performed using the LIMMA package to analyze for differences between conditions and the results were filtered for only those genes with false-discovery rate adjusted p values less than or equal to 0.05.

Mass cytometry: All mass cytometry data were collected on a CyTOF2 mass cytometer (Fluidigm) and analyzed using Cytobank. Mass cytometry data were analyzed using previously described methods, and sample staining and data collection was performed as previously described.

NSG xenograft model and BLI imaging: K562-expressing luciferase tumor cells (1×106) were injected intravenously (i.v.) via tail vein into 8-12-week-old male and female NOD-SCID-IL2Rγ^{-/-} (NSG) mice (The Jackson Laboratory, Bar Harbor, ME) on day 0. All mice were irradiated with 2.5 cGy 2 days before tumor injection. At day 3, BLI was performed to confirm leukemia cells engraftment. On Day 4, 5×10⁶ control (NK cells in 1 ng/mL IL-15) or NK cells activated with IL-12/15/18 (10 ng/mL IL-12, 50 ng/mL IL-15, 50 ng/mL IL-18), or 18/12/TxM (38 nM) were administered retro-orbitally to the mice (total 9-10 mice per group from 2 independent experiments). The mice were treated with rhIL-2 (50,000 IU per mouse) every other data and monitored weekly for tumor burden (BLI).

In vivo BLI imaging was performed on an IVIS 50, (1-60 sec exposure, bin8, FOV12 cm, open filter) (Xenogen, Alameda, CA). For this, mice were injected intraperitoneally with D-luciferin (150 mg/kg in PBS, Gold Biotechnology, St. Louis, MO) and imaged under anesthesia with isoflourane (2% vaporized in O₂). Total photon flux (photons/sec) was measured from fixed regions of interest over the entire mouse using the Living Image 2.6 software program.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

1. A method of generating a memory-like cytokine enhanced natural killer (M-CENK) cell, comprising:
obtaining a plurality of mononuclear cells, and contacting the plurality of mononuclear cells with a corticosteroid and an IL-15 or an agonist analog thereof;
incubating the plurality of mononuclear cells in the presence of the corticosteroid and the IL-15 or an agonist analog thereof to enrich the mononuclear cells in cytokine enhanced NK cells (CENK); and
inducing the enriched NK cells with a cytokine composition to generate the M-CENK cells, wherein the cytokine composition comprises IL-12 or an agonist analog thereof, IL-15 or an agonist analog thereof, and IL-18 or an agonist analog thereof.

2. The method of claim 1 wherein the plurality of mononuclear cells are cryopreserved before the step of incubating.

3. The method of claim 2 wherein the cryopreserved mononuclear cells are thawed, washed, and resuspended in a medium containing the corticosteroid and the IL-15 or agonist analog thereof.

4. The method of claim 1, wherein the step of incubating is performed over a period of between 14 and 21 days.

5. The method of claim 1, wherein the step of incubating is performed until the NK cells are enriched to at least 65% of all live cells.

6. (canceled)

7. The method of claim 1, wherein the enriched NK cells are induced over a period of between 12 and 16 hours.

8. The method of claim 1, wherein the corticosteroid is hydrocortisone and the IL-15 agonist analogue is N-803.

9. (canceled)

10. The method of claim 1, further comprising a step of harvesting the M-CENK cells and formulating the harvested M-CENK cells for infusion.

11. The method of claim 10 wherein the harvested M-CENK cells are cryopreserved before infusion.

12. (canceled)

13. A memory-like cytokine enhanced natural killer (M-CENK) cell produced by the method of claim 1.

14. A composition comprising a pharmaceutically acceptable carrier in combination with the M-CENK cell of claim 13.

15. The composition of claim 14 wherein the pharmaceutically acceptable carrier comprises a cryopreservation medium.

16. The composition of claim 14, wherein the pharmaceutically acceptable carrier is formulated for infusion.

17. The composition of claim 14, having a cell density of 0.5-1.5×10⁷ cells/mL.

18. The composition of claim 14, wherein the composition is a cryopreserved composition.

19. A method of treating an individual having cancer, comprising a step of administering the composition of claim 14 by transfusion.

20. The method of claim 19 wherein the composition treats cancer in the individual by killing cancer stem cells and mesenchymal cells.

21. (canceled)

22. (canceled)

23. The method of claim 1, wherein two of the cytokines comprising the cytokine composition comprise a fusion protein, wherein a protein portion having activity of the first cytokine is fused to a protein portion having activity of the second cytokine.

24. The method of claim 1, wherein the cytokine composition comprises a TxM fusion protein, wherein the TxM fusion protein comprises a protein portion having IL-12 activity, a protein portion having IL-15 activity, and a protein portion having IL-18 activity.
